(19) United States
(12) Patent Application Publication
(10) Pub. No.: US 2011/0256227 A1
(43) Pub. Date: Oct. 20, 2011

(54) CONTROLLED POLYPLEX ASSEMBLY

(75) Inventors: Janni Mirosevich, Tampa, FL (US); Kevin N. Sill, Tampa, FL (US)

(73) Assignee: Intezyne Technologies, Inc., Tampa, FL (US)

(21) Appl. No.: 13/087,339

(22) Filed: Apr. 14, 2011

Related U.S. Application Data
(60) Provisional application No. 61/324,133, filed on Apr. 14, 2010.

(51) Int. Cl. A61K 9/14; A61K 31/7105; B82Y 5/00

(52) U.S. Cl. 424/490; 514/44 R; 424/400; 977/773; 977/906

(57) ABSTRACT

The present invention provides polymers, compositions thereof, and polyplexes comprising said polymers. In particular, polyplexes comprising polycations, polyanions, and polynucleotides are provided. The invention further provides methods of making and using said polyplexes.

### CROSS REFERENCE TO RELATED APPLICATIONS

The present application claims priority to U.S. provisional patent application Ser. No. 61/324,133, filed Apr. 14, 2010, the entirety of which is hereby incorporated herein by reference.

### FIELD OF THE INVENTION

The present invention relates to the field of polymer chemistry and more particularly to polynucleotide containing polyplexes and uses thereof.

### BACKGROUND OF THE INVENTION

There are several key factors that limit the use of lipoplexes and polyplexes for in vivo gene delivery applications, particularly when systemic delivery is desired. These include instability of these electrostatic assemblies in high salt environments, irreversible protein binding to the complex that can alter their pharmacokinetic profile, and capture by RES due to excess positive charge. The covalent attachment of poly(ethylene glycol) (PEG) to gene carriers has been shown to address many of these limitiations by sterically shielding the complex from unwanted cellular and protein interactions as well as imparting the inherent, stealth properties of PEG. MacLachlan and coworkers have demonstrated that PEG-lipid conjugates, used in conjunction with traditional lipids, can dramatically improve the stability and circulation half-life of DNA-loaded lipoplexes (J. Control. Release, 2006, 112, 280). Similarly, Kissel and coworkers have developed PEG-modified PEI polyplexes that showed enhanced circulation lifetimes when compared to unmodified PEI polyplexes (Pharm. Res., 2002, 19, 810).

Another factor that largely remains unsolved for RNA containing polyplexes is the matter of particle size. For in vivo applications, it is highly desirable to have a particle size that is approximately 100 nm in diameter, more specifically from 20 nm to 200 nm in diameter. Below 20 nm, particles are readily removed from circulation by the kidneys, while particles above 200 nm are removed by the liver and spleen. Particles that have possess a size of 20-200 nm are able to avoid renal and RES (reticuloendothelial system) uptake and circulate for a much longer period of time, allowing the particles a greater probability of reaching the desired location within the body. Accordingly, it would be highly desirable to produce a RNA containing polyplex with a size of approximately 20-200 nm.

### BRIEF DESCRIPTION OF THE DRAWINGS

FIG. 1. Depicts the gel retardation results for Examples 5, 6, and 7.

FIG. 2. Depicts the dynamic light scattering results for Examples 5, 6, and 7.

FIG. 3. Depicts the transmission electron microscopy results for Examples 5, 6, and 7.

### DETAILED DESCRIPTION OF CERTAIN EMBODIMENTS OF THE INVENTION

### 1. General Description

Preparation of polyplexes comprising DNA can result in a small, uniform particle size. This phenomenon is due to the inherent characteristic for DNA to collapse upon itself, commonly referred to as compaction. Without wishing to be bound to any particular theory, it is believed that as plasmid DNA is complexed with a suitable cationic polymer, the DNA begins to compact into a tightly bound globular structure. Because anionic DNA collapses, it is possible to collapse the DNA such that the outside of the globular complex is fully covered with the cationic polymer. This compacting upon complexation leads to uniform particle sizes and a minimal amount of aggregation. In essence, the polyplex size is templated based upon the compaction of the DNA.

In contrast, RNA lacks an internal driving force for compaction. Rather, when RNA encounters a suitable polycation (i.e., for polyplex formation), an electrostatic interaction occurs between the anionic polynucleotide (i.e, the RNA) and the cationic polymer. However, because there is no driving force to sequester the negative charge towards the center of the polyplex, the negatively charged region of the resulting RNA complex can interact with a positively charged region of another complex, and so on. Thus, such RNA polyplexes tend to aggregate and result in particles of non-uniform size and particle diameters often in the micron range.

It has been surprisingly found that uniform particles of sub-micron size containing RNA were achieved by combining RNA with a suitable polyanion in the presence of a suitable cation for polyplex formation. Accordingly, in some embodiments, the present invention provides a sub-micron particle comprising RNA, a suitable polyanion, and a suitable polycation.

### 2. Definitions

Compounds of this invention include those described generally above, and are further illustrated by the embodiments, sub-embodiments, and species disclosed herein. As used herein, the following definitions shall apply unless otherwise indicated. For purposes of this invention, the chemical elements are identified in accordance with the Periodic Table of the Elements, CAS version, Handbook of Chemistry and Physics, 75^{th} Ed. Additionally, general principles of organic chemistry are described in “Organic Chemistry,” Thomas Sorrell, University Science Books, Sausalito: 1999, and “March's Advanced Organic Chemistry”, 5^{th} Ed., Ed.: Smith, M. B. and March, J., John Wiley & Sons, New York: 2001, the entire contents of which are hereby incorporated by reference.

As used herein, the term “portion” or “block” refers to a repeating polymeric sequence of defined composition. A portion or a block may consist of a single monomer or may be comprise of on or more monomers, resulting in a “mixed block”.

One skilled in the art will recognize that a monomer repeat unit is defined by parentheses depicted around the repeating monomer unit. The number (or letter representing a numerical range) on the lower right of the parentheses represents the number of monomer units that are present in the polymer chain. In the case where only one monomer represents the block (e.g. a homopolymer), the block will be denoted solely by the parentheses. In the case of a mixed block, multiple monomers comprise a single, continuous block. It will be understood that brackets will define a portion or block. For example, one block may consist of four individual monomers, each defined by their own individual set of parentheses and number of repeat units present. All four sets of parentheses will be enclosed by a set of brackets, denoting that all four of these monomers combine in random, or near random, order to comprise the mixed block. For clarity, the randomly mixed block of [BCADDCBADABCDABC] would be represented in shorthand by [(A)₄(B)₄(C)₄(D)₄].

As used herein, the term “polycation” or “cationic polymer” may be used interchangeably and refer to a polymer possessing a plurality of cationic charges. In some embodiments polycation also refers to a polymer that possess a plurality of functional groups that can be protonated to obtain a plurality of cationic charges. For clarity, a polymer that contains a plurality of amine functional groups will be referred to as a polycation or a cationic polymer within this application.

In some embodiments, a “suitable polycation” refers to any polycationic material that is capable with interacting with RNA. In some embodiments, a suitable polycation forms a polyplex with RNA. In some embodiments, a suitable polycation forms a sub-micron polyplex with RNA and a suitable polyanion. In some embodiments, a suitable polycation is a transfection agent. Exemplary suitable polycations also include poly(amines), poly(ketimines), poly(amino acids), and poly(guanidinium). In certain embodiments, a suitable polycation is selected from poly(alkylamines), poly(arylamines), poly(alkenylamines), and poly(alkynylamines), such as poly(imidazoles), poly(pyridines), poly(pyrimidines), poly(pyrazoles), poly(lysine), branched or linear poly(ethyleneimine), poly(histidine), poly(ornithine), poly(arginine), poly(asparginine), poly(glutamine), poly(tryptophan), poly(vinylpyridine), cationic guar gum, Oligofectamine® (from Invitrogen), polyfectamine® (from Qiagen), SuperFect® (from Qiagen), 293Fectin ((from Invitrogen), Cellfectin (from Invitrogen), DMRIE-C (from Invitrogen), FreeStyle (from Invitrogen), Lipofectamine 2000® (from Invitrogen), siPORT (from Invitrogen), Optifect (from Invitrogen), Neon (from Invitrogen), or salts and/or mixtures thereof.

As used herein, a “suitable polyanion” refers to any polyanionic material that has a potential driving force for compaction. Exemplary suitable polyanions include polynucleotides, polyelectrolytes, polyampholytes, poly(amino acids), poly(phosphonic acids), poly(phosphonates), poly(boronic acids), poly(boronates), polyphosphazines, and the like. Such suitable polyanions include any double stranded DNA (e.g., plasmid DNA), poly(styrene sulfonate), poly(acrylic acid), poly(acrylate), poly(aspartic acid), poly(glutamic acid), poly(aspartate), poly(glutamate), alginic acid, carboxymethylcellulose, alginates, poly(vinylbenzoate), poly(methacrylic acid), polyphosphonates, poly(vinylphosphonic acid), or salts and/or mixtures thereof.

As used herein, the term “RNA” refers to any single stranded polynucleotide. In some embodiments, RNA includes mRNA (messenger RNA), tRNA (transfer RNA), rRNA (ribosomal RNA), snRNA (small nuclear RNA), siRNA (short interfering RNA), miRNA (micro-RNA), shRNA (short hair-pin RNA), asRNA (antisense RNA), tmRNA (transfer messenger RNA), piRNA (Piwi-interacting RNA), and rasiRNA (repeat associated short interfering RNA).

As used herein, the term “controlled assembly” refers to formation of a polynucleotide polyplex in a controlled fashion to reduce, or even eliminate, particle aggregation.

As used herein, the phrase N to P (N/P or N:P) refers to the ratio of protonatable nitrogens (N) to negatively charged phosphate groups in the DNA or RNA backbone (P).

As used herein, the term “D,L-mixed poly(amino acid)” refers to a poly(amino acid) wherein the poly(amino acid) consists of a mixture of amino acids in both the D- and L-configurations. It is well established that homopolymers and copolymers of amino acids, consisting of a single stereoisomer, may exbibit secondary structures such as the α-helix or β-sheet. See α-Aminoacid-N-Caroboxy-Anhydrides and Related Heterocycles, H. R. Kricheldorf, Springer-Verlag, 1987. For example, poly(L-benzyl glutatmate) typically exhibits an α-helical conformation; however this secondary structure can be disrupted by a change of solvent or temperature (see Advances in Protein Chemistry XVI, P. times and P. Doty, Academic Press, New York 1961). The secondary structure can also be disrupted by the incorporation of structurally dissimilar amino acids such as β-sheet forming amino acids (e.g. proline) or through the incorporation of amino acids with dissimilar stereochemistry (e.g. mixture of D and L stereoisomers), which results in poly(amino acids) with a random coil conformation. See Sakai, R.; Ikeda; S.; Isemura, T. Bull Chem. Soc. Japan 1969, 42, 1332-1336, Paolillo, L.; Temussi, P. A.; Bradbury, E. M.; Crane-Robinson, C. Biopolymers 1972, 11, 2043-2052, and Cho, I.; Kim, J. B.; Jung, H. J. Polymer 2003, 44, 5497-5500.

As used herein, the term “tacticity” refers to the stereochemistry of the poly(amino acid). A poly(amino acid) block consisting of a single stereoisomer (e.g. all L isomer) is referred to as “isotactic”. A poly(amino acid) consisting of a random incorporation of D and L amino acid monomers is referred to as an “atactic” polymer. A poly(amino acid) with alternating stereochemistry (e.g. . . . DLDLDL . . . ) is referred to as a “syndiotactic” polymer. Polymer tacticity is described in more detail in “Principles of Polymerization”, 3rd Ed., G. Odian, John Wiley & Sons, New York: 1991, the entire contents of which are hereby incorporated by reference.

As used herein, the term “targeting group” refers to any molecule, macromolecule, or biomacromolecule that selectively binds to receptors that are expressed or over-expressed on specific cell types. Targeting groups are well known in the art and include those described in International application publication number WO 2008/134731, published Nov. 6, 2008, the entirety of which is hereby incorporated by reference. In some embodiments, the targeting group is a moiety selected from folate, a Her-2 binding peptide, a urokinase-type plasminogen activator receptor (uPAR) antagonist, a CXCR4 chemokine receptor antagonist, a GRP78 peptide antagonist, an RGD peptide, an RGD cyclic peptide, a luteinizing hormone-releasing hormone (LHRH) antagonist peptide, an aminopeptidase targeting peptide, a brain homing peptide, a kidney homing peptide, a heart homing peptide, a gut homing peptide, an integrin homing peptide, an angiogencid tumor endothelium homing peptide, an ovary homing peptide, a uterus homing peptide, a sperm homing peptide, a microglia homing peptide, a synovium homing peptide, a urothelium homing peptide, a prostate homing peptide, a lung homing peptide, a skin homing peptide, a retina homing peptide, a pancreas homing peptide, a liver homing peptide, a lymph node homing peptide, an adrenal gland homing peptide, a thyroid homing peptide, a bladder homing peptide, a breast homing peptide, a neuroblastoma homing peptide, a lymphona homing peptide, a muscle homing peptide, a wound vasculature homing peptide, an adipose tissue homing peptide, a virus binding peptide, or a fusogenic peptide.

The term “oligopeptide”, as used herein refers to any peptide of 2-65 amino acid residues in length. In some embodiments, oligopeptides comprise amino acids with natural amino acid side-chain groups. In some embodiments, oligopeptides comprise amino acids with unnatural amino acid side-chain groups. In certain embodiments, oligopeptides are 2-50 amino acid residues in length. In certain embodiments, oligopeptides are 2-40 amino acid residues in length. In some embodiments, oligopeptides are cyclized variations of the linear sequences. In other embodiments, oligopeptides are 3-15 amino acid residues in length.

The term “substantially free of”, as used herein, means containing no more than an insignificant amount. In some embodiments, a composition or entity is “substantially free of” a recited element if it contains less than 5%, 4%, 3%, 2%, or 1%, by weight of the element(s). In some embodiments, the composition or entity contains less than 0.9%, 0.8%, 0.7%, 0.6%, 0.5%, 0.4%, 0.3%, 0.2%, 0.1% or less of the recited element(s). In some embodiments, the composition or entity contains an undetectable amount of the recited element(s).

### 3. Description of Exemplary Embodiments

As described generally above, in some embodiments, the present invention provides a sub-micron particle comprising RNA, a suitable polyanion, and a suitable polycation. In certain embodiments, the present invention provides a polyplex comprising RNA, a suitable polyanion, and a suitable polycation.

In some embodiments, the ratio of RNA to polyanion is 1:1, 2:1, 3:1, or 4:1.

In certain embodiments, the N/P ratio is >1. In some embodiments, the N/P ratio is about 2 to about 50. In some embodiments, the N/P ratio is about 2, about 5, about 10, about 20, about 40, or about 50. In some embodiments, the N/P ratio is about 10.

In some embodiments, a provided polyplex is about 20 to about 200 nm, about 50 to about 100, about 100 to about 150, about 60 to about 80, or about 100 to about 120. In some embodiments, a provided polyplex is less than about 1 μm.

In some embodiments, a suitable polycation refers to any polycationic material that is capable with interacting with RNA. In some embodiments, a suitable polycation forms a polyplex with RNA. In some embodiments, a suitable polycation forms a sub-micron polyplex with RNA and a suitable polyanion. In some embodiments, a suitable polycation is a transfection agent. Exemplary suitable polycations also include poly(amines), poly(ketimines), poly(amino acids), poly(guanidinium), poly(alkylamines), poly(arylamines), poly(alkenylamines), and poly(alkynylamines), such as poly(imidazoles), poly(pyridines), poly(pyrimidines), poly(pyrazoles), poly(lysine), branched or linear poly(ethyleneimine), poly(histidine), poly(ornithine), poly(arginine), poly(asparginine), poly(glutamine), poly(tryptophan), poly(vinylpyridine), cationic guar gum, Oligofectamine® (from Invitrogen), polyfectamine® (from Qiagen), SuperFect® (from Qiagen), 293Fectin ((from Invitrogen), Cellfectin (from Invitrogen), DMRIE-C (from Invitrogen), FreeStyle (from Invitrogen), Lipofectamine 2000® (from Invitrogen), siPORT (from Invitrogen), Optifect (from Invitrogen), Neon (from Invitrogen), or salts and/or mixtures thereof.

In certain embodiments, the present invention provides a method for controlling the size of an RNA-containing complex comprising combining the RNA with a suitable polyanion.

In certain embodiments, the polyanion is a polynucleotide. In some embodiments, a polynucleotide is a short interfering RNA (siRNA), a microRNA (miRNA), a plasmid DNA (pDNA), a short hairpin RNA (shRNA), messanger RNA (mRNA), antisense RNA (asRNA), to name a few, and encompasses both the nucleotide sequence and any structural embodiments thereof, such as double stranded, single stranded, helical, hairpin, etc. In some embodiments, the polyanion is DNA. In some embodiments, the polyanion is plasmid DNA. In certain embodiments, the polyanion is an amphiphilic polyanion. In other embodiments, the polyanion is poly(styrene sulfonate). In yet other embodiments, the polyanion is poly(acrylic acid).

In some embodiments, the present invention provides a polyplex comprising an RNA and a polyanion. In certain embodiments, the polyanion is selected from polynucleotides, polyelectrolytes, polyampholytes, poly(amino acids), poly(phosphonic acids), poly(phosphonates), poly(boronic acids), poly(boronates), polyphosphazines, and salts and/or mixtures thereof. In certain embodiments, the suitable polyanion is any double stranded DNA (e.g., plasmid DNA), poly(styrene sulfonate), poly(acrylic acid), poly(acrylate), poly(aspartic acid), poly(glutamic acid), poly(aspartate), poly(glutamate), alginic acid, carboxymethylcellulose, alginates, poly(vinylbenzoate), poly(methacrylic acid), polyphosphonates, poly(vinylphosphonic acid), or a salt and/or mixture thereof.

As described generally herein, in some embodiments the present invention provides a polynucleotide polyplex comprising RNA, a polyanion, and a cationic polymer comprising a poly(amino acid) block. In certain embodiments, the cationic polymer may be comprised of a mixed poly(amino acid) block. In some embodiments, the cationic polymer is comprised of a poly(amino acid) block where all the amino acid units are in the L-configuration. In other embodiments, the cationic polymer is comprised of a poly(amino acid) block where the amino acid units are a mixture of D and L configurations.

In certain embodiments, a provided polycation is suitable for RNA encapsulation (i.e, polyplex formation).

In certain embodiments, the cationic polymer described above contains a mixture of primary and secondary amine groups on the side chain of the poly(amino acid). One of ordinary skill in the art will recognize that primary amine groups interact with phosphates in the polynucleotide to form the polyplex, whereas secondary amine groups function as a buffering group, or proton sponge, which aids in endosomal escape via endsome disruption. Ideally, one would select the optimum number of primary and secondary amines to both complex the polynucleotide and allow for sufficient endosomal escape, while limiting cytotoxicity.

In certain embodiments, the polycation is a compound of formula I, or a salt thereof:

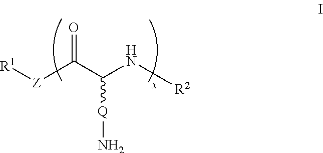

wherein:
   x is 10-250;
   each Q group is independently selected from a valence bond or a bivalent, saturated or unsaturated, straight or branched C₁₋₂₀ alkylene chain, wherein 0-9 methylene units of Q are independently replaced by -Cy-, —O—, —NH—, —S—, —OC(O)—, —C(O)O—, —C(O)—, —SO—, —SO₂—, —NHSO₂—, —SO₂NH—, —NHC(O)—, —C(O)NH—, —OC(O)NH—, or —NHC(O)O—, wherein:
      -Cy- is an optionally substituted 5-8 membered bivalent, saturated, partially unsaturated, or aryl ring having 0-4 heteroatoms independently selected from nitrogen, oxygen, or sulfur, or an optionally substituted 8-10 membered bivalent saturated, partially unsaturated, or aryl bicyclic ring having 0-5 heteroatoms independently selected from nitrogen, oxygen, or sulfur;
   Z is a valence bond or a bivalent, saturated or unsaturated, straight or branched C₁₋₁₂ hydrocarbon chain, wherein 0-6 methylene units of Q are independently replaced by -Cy-, —O—, —NH—, —S—, —OC(O)—, —C(O)O—, —C(O)—, —SO—, —SO₂—, —NHSO₂—, —SO₂—, —NHC(O)—, —C(O)NH—, —OC(O)NH—, or —NHC(O)O—, wherein:
      -Cy- is an optionally substituted 5-8 membered bivalent, saturated, partially unsaturated, or aryl ring having 0-4 heteroatoms independently selected from nitrogen, oxygen, or sulfur, or an optionally substituted 8-10 membered bivalent saturated, partially unsaturated, or aryl bicyclic ring having 0-5 heteroatoms independently selected from nitrogen, oxygen, or sulfur;
   R¹ is hydrogen, —N₃, —CN, a mono-protected amine, a di-protected amine, a protected aldehyde, a protected hydroxyl, a protected carboxylic acid, a protected thiol, a 9-30 membered crown ether, or an optionally substituted group selected from aliphatic, a 5-8 membered saturated, partially unsaturated, or aryl ring having 0-4 heteroatoms independently selected from nitrogen, oxygen, or sulfur, an 8-10 membered saturated, partially unsaturated, or aryl bicyclic ring having 0-5 heteroatoms independently selected from nitrogen, oxygen, or sulfur, or a detectable moiety or an oligopeptide targeting group;
   R² is selected from hydrogen, an optionally substituted aliphatic group, an acyl group, a sulfonyl group, or a fusogenic peptide.

In certain embodiments, the x group is about 10 to about 250. In certain embodiments, the x group is about 25. In other embodiments x is about 10 to about 50. In other embodiments, x is about 50. According to yet another embodiment, x is about 75. In other embodiments, x is about 100. In certain embodiments, x is about 40 to about 80. In other embodiments, x is selected from 10±5, 15±5, 25±5, 50±5, 75±10, 100±10, or 125±10.

In certain embodiments, Z is a —NH—group. In certain embodiments, Z is a valence bond. In some embodiments, Z is a bivalent, saturated or unsaturated, straight or branched C₁₋₈ hydrocarbon chain, wherein 0-3 methylene units are independently replaced by —O—, —NH—, —S—, —OC(O)—, —C(O)O—, —C(O)—, —NHC(O)—, or —C(O)NH—.

One skilled in the art will recognize that the stereochemistry of the poly(amino acid) represented in Formula I is undefined. It is understood that this depiction can represent an all L conformation, an all D conformation, or any random mixture of D and L isomers.

In some embodiments, R¹ is an optionally substituted aliphatic group. In certain embodiments, the R¹ group is a saturated or unsaturated C₁₋₁₂ alkyl chain. In other embodiments, the R¹ group is a pentyl group. In other embodiments, the R¹ group is a hexyl group. In other embodiments, the R¹ group is a hydrogen atom. In other embodiments, the R¹ group is a quaternized triethylamine group. In some embodiments, where Z comprises an amine, R¹ is a suitable amine protecting group. In some embodiments, where Z comprises a hydroxyl, R¹ is a suitable hydroxyl protecting group. In some embodiments, R¹ is or comprises an azide group. In some embodiments, R¹ is or comprises an alkynyl group.

In certain embodiments, the R² group is an acetyl group. In some embodiments, the R² group is a hydrogen atom. In some embodiments, R² is acyl. In some embodiments, R² is a fusogenic peptide.

In certain embodiments, the Q group is a chemical moiety representing an oligomer of ethylene amine, —(NH₂—CH₂—CH₂)—. In some embodimnets, Q is a bivalent, saturated or unsaturated, straight or branched C₁₋₂₀ alkylene chain, wherein 0-9 methylene units of Q are independently replaced by —NH—, —C(O)—, —NHC(O)—, or —C(O)NH—. In certain embodiments, Q is a branched alkylene chain wherein one or more methine carbons is replaced with a nitrogen atom to form a trivalent amine group. Specific examples of Q groups can be found in Table 1a, Table 1b, and Table 1c.

TABLE 1a

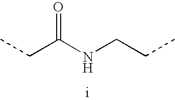

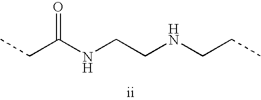

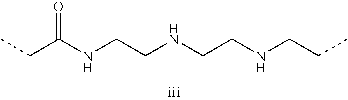

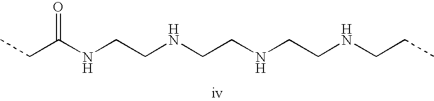

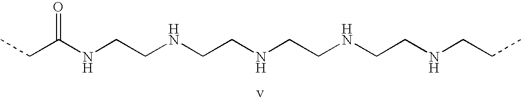

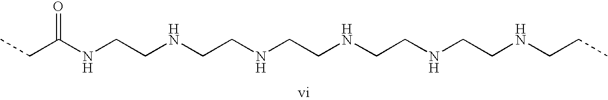

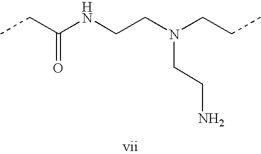

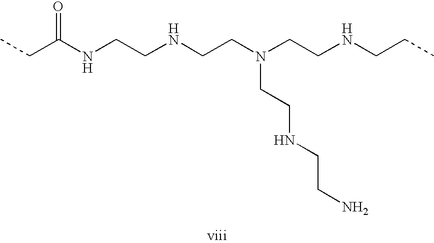

TABLE 1b

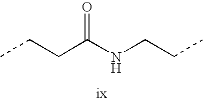

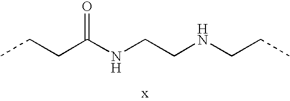

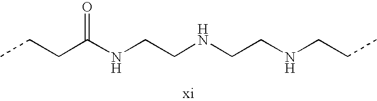

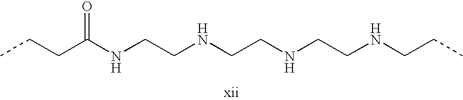

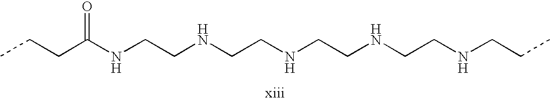

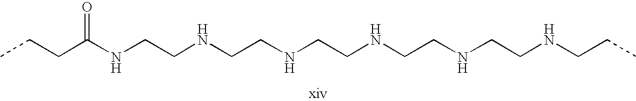

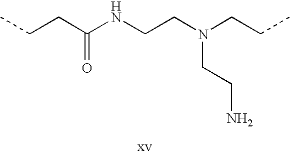

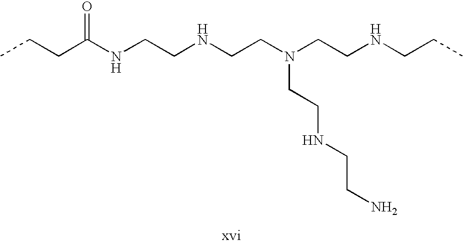

TABLE 1c.

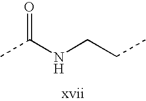

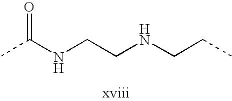

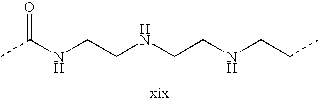

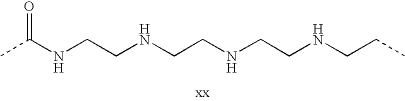

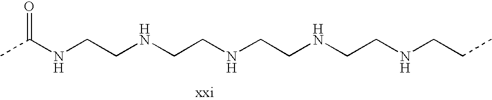

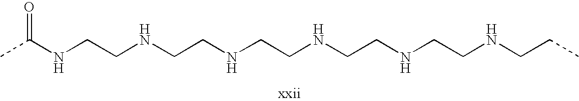

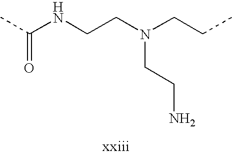

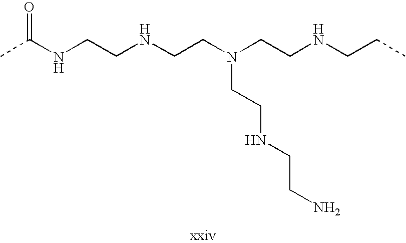

In some embodiments, the polycation is a compound of formula I-a, or a salt thereof:

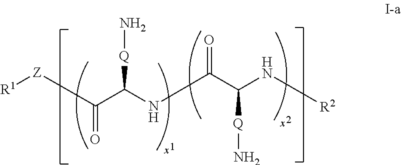

wherein:
   x is 0 to 250,
   x² is 0 to 250, provided that z¹ and z² are not simultaneously zero such that the sum of z¹ and z² is at least 5;
   each of R¹, Q, Z, x¹, x² and R² is as defined above and as described in classes and subclasses herein, both singly and in combination.

In certain embodiments, x¹ is about 10 to about 250. In certain embodiments, x¹ is about 25. In certain embodiments, x¹ is about 10. In certain embodiments, x¹ is about 15. In certain embodiments, x¹ is about 20. In other embodiments x¹ is about 10 to about 50. In other embodiments, x¹ is about 50. According to yet another embodiment, x¹ is about 75. In other embodiments, x¹ is about 100. In other embodiments, x¹ is selected from 10±5, 15±5, 25±5, 50±5, 75±10, 100±10, or 125±10.

In certain embodiments, the x² group is about 10 to about 250. In certain embodiments, the x² group is about 25. In certain embodiments, the x² group is about 10. In certain embodiments, the x² group is about 15. In certain embodiments, the x² group is about 20. In other embodiments x² is about 10 to about 50. In other embodiments, x² is about 50. According to yet another embodiment, x² is about 75. In other embodiments, x² is about 100. In other embodiments, x² is selected from 10±5, 15±5, 25±5, 50±5, 75±5, 100±10, or 125±10.

It will be appreciated by one skilled in the art that each of formulae I and I-a represent a polyamine, or a salt thereof. When either of formulae I or I-a is dissolved in an aqueous solution at pH 4-9, it will be appreciated that a plurality of the amino groups will exist as an ammonium salt (—NH₃⁺) with a suitable anion, while other amino groups will exist as the free base (—NH₂). One skilled in the art will readily recognize that the ratio between the protonated ammonium salt and the free base is heavily influenced by pH, as lower pH values will result in a high population of the ammonium salt and high pH values will result in a high population of the free base. Thus, it is contemplated that the polyamines of formulae I and I-a exist as a polycation in aqueous solution.

As generally described above, a suitable salt describes any anion capable of reacting with an amine to form an ammonium salt. Examples include, but are not limited to, chloride, bromide, iodide, fluoride, acetate, formate, trifluoroacetate, difluoroacetate, trichloroacetate, and phosphate.

B. Controlled Polyplex Assembly

In some embodiments, the present invention provides the controlled assembly of a polyplex formed by the combination of a cationic polymer, a polyanion, and a polynucleotide.

In water, cationic copolymers co-assemble with polynucleotides through electrostatic interactions between the cationic side chains of the polymer and the anionic phosphates of the polynucleotide to form a polyplex. In some cases, the number of phosphates on the polynucleotides may exceed the number of cationic charges on the multiblock copolymer. It will be appreciated that multiple polymers may be used to achieve charge neutrality (i.e. N/P=1) between the polymer and encapsulated polynucleotide. It will also be appreciated that when an excess of polymer is used to encapsulate a polynucleotide, the polymer/polynucleotide complex can possess an overall positive charge (i.e. N/P>1).

In certain embodiments, an encapsulated polynucleotide is capable of silencing gene expression via RNA interference (RNAi). As defined herein, RNAi is cellular mechanism that suppresses gene expression during translation and/or hinders the transcription of genes through destruction of messenger RNA (mRNA). Without wishing to be bound by any particular theory, it is believed that endogenous double-stranded RNA located in the cell are processed into 20-25 nt short-interfering RNA (siRNA) by the enzyme Dicer. siRNA subsequently binds to the RISC complex (RNA-induced silencing nuclease complex), and the guide strand of the siRNA anneals to the target mRNA. The nuclease activity of the RISC complex then cleaves the mRNA, which is subsequently degraded (Nat. Rev. Mol. Cell Biol., 2007, 8, 23).

In some embodiments, the RNA is siRNA. In some embodiments, siRNA is a linear, double-stranded RNA that is 20-25 nucleotides (nt) in length and possesses a 2 nt, 3′ overhang on each end which can induce gene knockdown in cell culture or in vivo via RNAi. In certain embodiments, the encapsulated siRNA suppresses disease-relevant gene expression in cell culture, animals, or humans.

In certain embodiments, the RNA is a short-hairpin RNA (shRNA). As used herein, shRNA is a linear, double-stranded RNA, possessing a tight hairpin turn, which is synthesized in cells through transfection and expression of a exogenous pDNA. Without wishing to be bound by any particular theory, it is believed that the shRNA hairpin structure is cleaved to produce siRNA, which mediates gene silencing via RNA interference. In certain embodiments, the encapsulated shRNA suppresses gene expression in cell culture, animals, or humans that are responsible for a disease via RNAi.

In certain embodiments, the RNA is a microRNA (miRNA). As used herein, miRNA is a linear, single-stranded RNA that ranges between 21-23 nt in length and regulates gene expression via RNAi (Cell, 2004, 116, 281). In certain embodiments, an encapsulated miRNA suppresses gene expression in cell culture, animals, or humans that are responsible for a disease via RNAi.

In another embodiment, the RNA is a messenger RNA (mRNA). As used herein, mRNA is defined as a linear, single stranded RNA molecule, which is responsible for translation of genes (from DNA) into proteins. In certain embodiments, the encapsulated mRNA is encoded from a plasmid cDNA to serve as the template for protein translation. In certain embodiments, an encapsulated mRNA translates therapeutic proteins, in vitro and/or in vivo, which can treat disease.

In certain embodiments, the RNA is an antisense RNA (asRNA). As used herein, asRNA is a linear, single-stranded RNA that is complementary to a targeted mRNA located in a cell. Without wishing to be bound by any particular theory, it is believed that asRNA inhibits translation of a complementary mRNA by pairing with it and obstructing the cellular translation machinery. It is believed that the mechanism of action for asRNA is different from RNAi because the paired mRNA is not destroyed. In certain embodiments, an encapsulated asRNA suppresses gene expression in cell culture, animals, or humans that are responsible for a disease by binding mRNA and physically obstructing translation.

In certain embodiments, the present invention provides a polyplex having a RNA encapsulated therein, comprising a cationic polymer of formula I or I-a, or a salt thereof, and a polyanion.

In some embodiments, the present invention provides a sub-micron polyplex having a RNA encapsulated therein, comprising a cationic polymer of formula I or I-a, or a salt thereof, and a polyanion.

In some embodiments, the present invention provides a sub-micron polyplex having a RNA encapsulated therein, comprising a cationic polymer of formula I or I-a, or a salt thereof, and a DNA.

In some embodiments, the present invention provides a polyplex comprising RNA, a cationic polymer of formula I or I-a, or a salt thereof, and a polyanion, wherein each variable is as defined and described herein, both singly and in combination.

In some embodiments, the present invention provides a polyplex comprising RNA, poly(lysine), and DNA.

In some embodiments, the present invention provides a PEGylated polyplex comprising RNA, poly(lysine), and DNA.

In some embodiments, the present invention provides a targeted polyplex comprising RNA, poly(lysine), and DNA.

In some embodiments, the present invention provides a polyplex comprising RNA, poly(ethylenediamine), and DNA.

In some embodiments, the present invention provides a PEGylated polyplex comprising RNA, poly(ethylenediamine), and DNA.

In some embodiments, the present invention provides a targeted polyplex comprising RNA, poly(ethylenediamine), and DNA.

In some embodiments, the present invention provides a polyplex, comprising RNA, a suitable transfection agent, and DNA.

In some embodiments, the present invention provides a polyplex, comprising PEGylated RNA, a suitable transfection agent, and DNA.

In some embodiments, the present invention provides a targeted polyplex, comprising RNA, a suitable transfection agent, and DNA.

In certain embodiments, the present invention provides a polyplex as defined and described in classes and subclasses herein, both singly and in combination, wherein the polyplex is substantially free of cellular components other than those encapsulated. In certain embodiments, the polyplex is substantially free of polypeptides, oligopeptides, and polynucleotides, other than those encapsulated. It will be appreciated that “other than those encapsulated” includes polyanions or polycations which form the polyplex and/or function to compact RNA; however, extraneous cellular components which do not form the polyplex and/or function to compact RNA are excluded.

In certain embodiments, a provided polyplex has a size of about 20 nm to about 200 nm. In some embodiments, a polyplex is about 20 nm to about 100 nm. In some embodiments, a polyplex is about 20 nm to about 50 nm. In some embodiments, a polyplex is about 50 nm to about 200 nm. In some embodiments, a polyplex is about 50 nm to about 100 nm. In some embodiments, a polyplex is about 80 nm. In some embodiments, a polyplex is about 100 nm.

In some embodiments, the present invention provides a composition of polyplexes characterized in that, on average in the composition, a polyplex is about 20 nm to about 200 nm. In some embodiments, the present invention provides a composition of polyplexes characterized in that, on average in the composition, a polyplex is about 20 nm to about 100 nm. In some embodiments, the present invention provides a composition of polyplexes characterized in that, on average in the composition, a polyplex is about 20 nm to about 50 nm. In some embodiments, the present invention provides a composition of polyplexes characterized in that, on average in the composition, a polyplex is about 50 nm to about 200 nm. In some embodiments, the present invention provides a composition of polyplexes characterized in that, on average in the composition, a polyplex is about 50 nm to about 100 nm. In some embodiments, the present invention provides a composition of polyplexes characterized in that, on average in the composition, a polyplex is about 80 nm. In some embodiments, the present invention provides a composition of polyplexes characterized in that, on average in the composition, a polyplex is about 100 nm.

C. Polyplex PEGylation

In certain embodiments, the present invention provides a polyplex formed by the combination of an RNA, a cationic polymer, and a polyanion, followed by the covalent attachment of PEG to the polyplex to form a PEG-conjugated polyplex. In some embodiments, the present invention provides a PEGylated polyplex comprising RNA, a cationic polymer, and a polyanion.

One of ordinary skill in the art will recognize that multiple avenues to conjugate the PEG onto the polyplex exist. Generally, excess amines present within the polyplex will react with suitable electrophiles to form covalent bonds. Suitable electrophiles include, but are not limited to, maleimides, activated esters, esters, and aldehydes. It is also important to recognize that the pH of the solution will affect the reactivity of the excess amines present within the polyplex. At low pH, the amines will predominately exist as an ammonium salt, and the reaction rate of the ammonium salt with the electrophile will be very low. However, as the pH approaches basic conditions (>7), the amines will have a higher percentage of free amine compared to ammonium salts. When the percentage of free amines increases, the reaction rate with a suitable electrophile will also increase. Thus, it is advantageous to select a pH that allows for the highest reaction rate (basic pH) without causing an adverse effect to the polynucleotide. In some embodiments, the pH of the PEGylation reaction solution is 4.0-9.0. In some embodiments, the pH of the PEGylation reaction solution is 5.0-6.0. In other embodiments, the pH of the PEGylation reaction solution is 6.0-7.0. In some embodiments, the pH of the PEGylation reaction solution is 7.0-8.0. In yet other embodiments, the pH of the PEGylation reaction solution is about 7.0. In another embodiment, the pH of the PEGylation reaction solution is about 7.5. In yet another embodiments, the pH of the PEGylation reaction solution is about 7.4.

In certain embodiments, the present invention provides a polyplex comprising RNA, a polyanion, and a cationic polymer of formula II or a salt thereof:

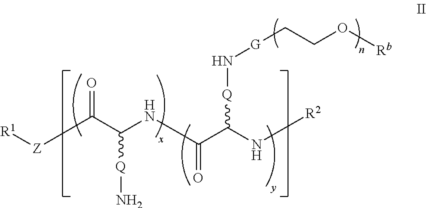

wherein each of R¹, Q, Z, x, and R² is as defined above and as described in classes and subclasses herein, both singly and in combination;
   y is 1-200;
   each n is independently 40-500;
   each G is independently a valence bond or a bivalent, saturated or unsaturated, straight or branched C₁₋₁₂ hydrocarbon chain, wherein 0-6 methylene units of Q are independently replaced by -Cy-, —O—, —NH—, —S—, —OC(O)—, —C(O)O—, —C(O)—, —SO—, —SO₂—, —NHSO₂—, —SO₂NH—, —NHC(O)—, —C(O)NH—, —OC(O)NH—, or —NHC(O)O—, wherein:
      -Cy- is an optionally substituted 5-8 membered bivalent, saturated, partially unsaturated, or aryl ring having 0-4 heteroatoms independently selected from nitrogen, oxygen, or sulfur, or an optionally substituted 8-10 membered bivalent saturated, partially unsaturated, or aryl bicyclic ring having 0-5 heteroatoms independently selected from nitrogen, oxygen, or sulfur;
   each R^{b} is independently —CH₃, a saturated or unsaturated alkyl moiety, an alkyne containing moiety, an azide containing moiety, a protected amine moiety, an aldehyde or protected aldehydes containing moiety, a thiol or protected thiol containing moiety, a cyclooctyne containing moiety, difluorocyclooctyne containing moiety, a nitrile oxide containing moiety, an oxanorbornadiene containing moiety, or an alcohol or protected alcohol containing moiety.

In some embodiments, the present invention provides a polyplex comprising RNA, a polyanion, and a cationic polymer of formula II, or a salt thereof, wherein each variable is as defined and described herein, both singly and in combination.

In certain embodiments, y is about 1 to about 200. In certain embodiments, y is about 25. In certain embodiments, y is about 10. In certain embodiments, y is about 20. In certain embodiments, y is about 15. In other embodiments y is about 1 to about 25. In other embodiments, y is about 50. According to yet another embodiment, y is about 25-75. In other embodiments, y is about 100. In other embodiments, y is selected from 10±5, 15±5, 25±5, 50±5, 75±10, 100±10, or 125±10.

As defined generally above, each n is independently 40-500. In certain embodiments n is about 225. In some embodiments, n is about 275. In other embodiments, n is about 110. In other embodiments, n is about 40 to about 60. In other embodiments, n is about 60 to about 90. In still other embodiments, n is about 90 to about 150. In other embodiments, n is about 150 to about 200. In some embodiments, n is about 200 to about 300, about 300 to about 400, about 400 to about 500. In still other embodiments, n is about 250 to about 280. In other embodiments, n is about 300 to about 375. In other embodiments, n is about 400 to about 500. In certain embodiments, n is selected from 50±10. In other embodiments, n is selected from 80±10, 115±10, 180±10, 225±10, 275±10, or 450±10.

In some embodiments, R^{b} is an optionally substituted aliphatic group containing an alkyne. In some embodiments, R^{b} is an optionally substituted aliphatic group containing an azide. In some embodiments, R^{b} is an optionally substituted aliphatic group containing an aldehyde or protected aldehyde. In some embodiments, R^{b} is an optionally substituted aliphatic group containing a thiol or protected thiol. In some embodiments, R^{b} is an optionally substituted aliphatic group containing a cyclooctyne group. In some embodiments, R^{b} is an optionally substituted aliphatic group containing a difluorocyclooctyne group. In some embodiments, R^{b} is an optionally substituted aliphatic group containing a oxanobornadiene group. In certain embodiments, R^{b} is —CH₂CH₂N₃. In other embodiments, R^{b} is —CH₃. In some embodiments, a polymer chain comprises a mixture of —CH₂CH₂N₃ and —CH₃ groups at the R⁶ position.

In certain embodiments, the G group is a valence bond. In other embodiments, the G group comprises a carbonyl group. In other embodiments, the G group is represented by a moiety in Table 2.

TABLE 2.s

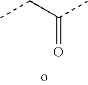

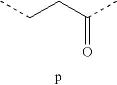

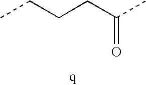

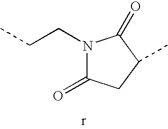

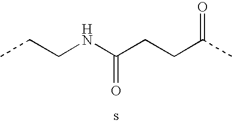

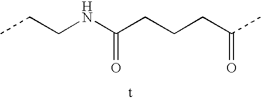

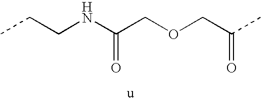

In certain embodiments, the present invention provides method of preparation for a PEG-conjugated polyplex comprising an RNA, a polyanion, and a cationic polymer of formula II or a salt thereof comprising the steps of:
(1) providing a polyplex having an RNA and a polyanion encapsulated therein, comprising a cationic polymer of formula I;
(2) optionally adjusting the pH of the polyplex solution to pH 4.0-9.0; and
(3) conjugating a compound of formula IV to the polyplex by reaction of the electrophile of formula IV and an amine group of formula I,

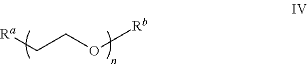

wherein each of R^{b} is as defined above and as described in classes and subclasses herein, both singly and in combination; and
R^{a} is or comprises a suitable electrophile;
n is 10-1000.

As generally described above, an electrophile of R^{a} is generally described as a moiety capable of reacting with a nucleophile to form a new covalent bond. In certain embodiments, a suitable electrophile is one that is capable of reacting with an amine derivative. Suitable electrophiles include, but are not limited to maleimide derivatives, activated ester moieties, esters, and aldehyde moieties.

As defined generally above, n is 40-500. In certain embodiments n is about 225. In some embodiments, n is about 275. In other embodiments, n is about 110. In other embodiments, n is about 40 to about 60. In other embodiments, n is about 60 to about 90. In still other embodiments, n is about 90 to about 150. In other embodiments, n is about 150 to about 200. In some embodiments, n is about 200 to about 300, about 300 to about 400, about 400 to about 500. In still other embodiments, n is about 250 to about 280. In other embodiments, n is about 300 to about 375. In other embodiments, n is about 400 to about 500. In certain embodiments, n is selected from 50±10. In other embodiments, n is selected from 80±10, 115±10, 180±10, 225±10, 275±10, or 450±10.

It will be appreciated by one skilled in the art that the copolymer of formula II represents a random, mixed copolymer of free amines or ammonium salts and amines that have reacted with a polymer of formula IV to provide a covalent bond attaching the grafted PEG chain to the poly(amino acid) backbone. Thus, a mixture of free amines or ammonium salts and PEG chains now represents the side chains of the poly(amino acid) copolymer. It will be appreciated that if and only if the x group of formula II is zero, then each and every amine would have reacted with a compound of formula IV and no free amine or ammoniums salts would exist in formula II. In some embodiments, x is zero. In other embodiments, x is nonzero.

Exemplary compounds of formula IV can be found in Table 3a and 3b, wherein each n is independently 40-500.

TABLE 3a

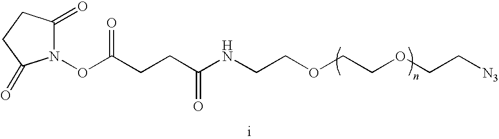

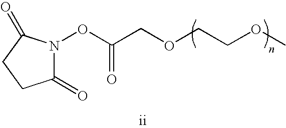

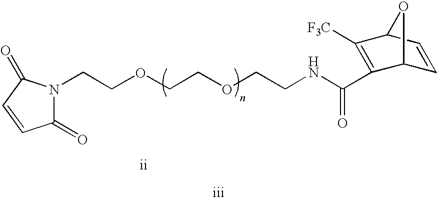

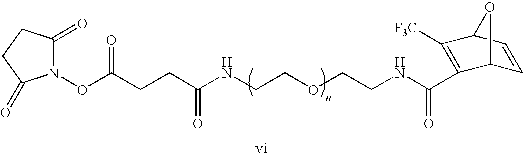

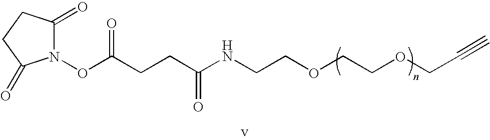

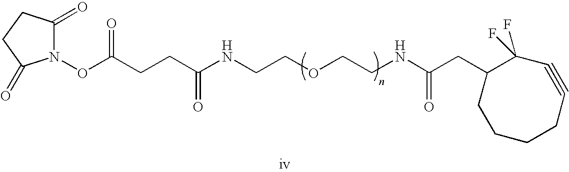

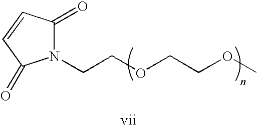

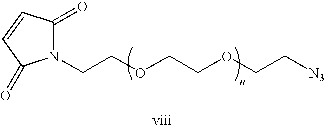

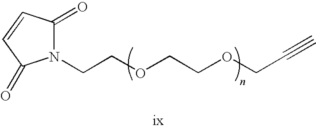

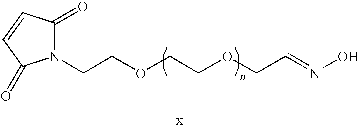

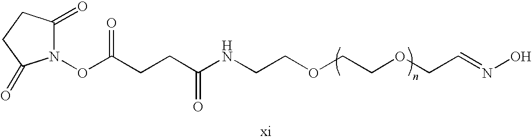

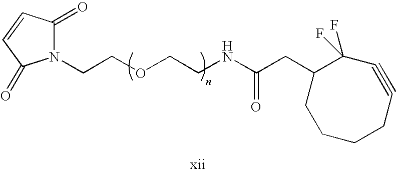

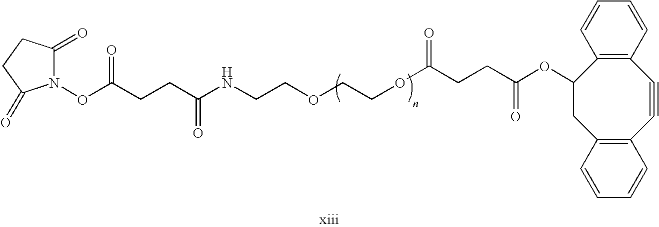

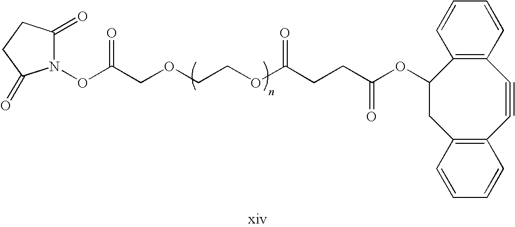

TABLE 3b

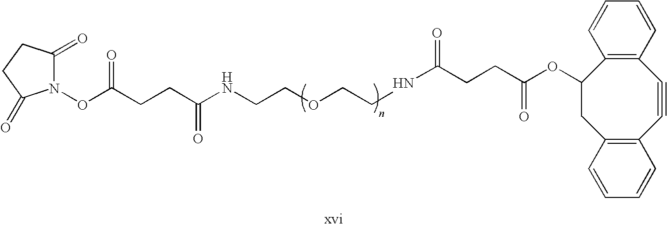

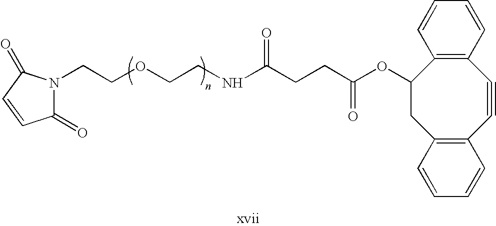

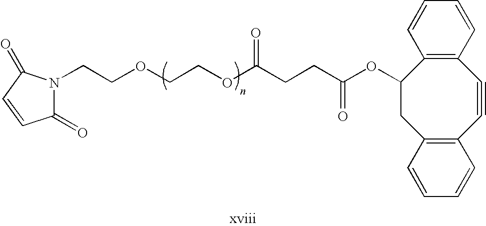

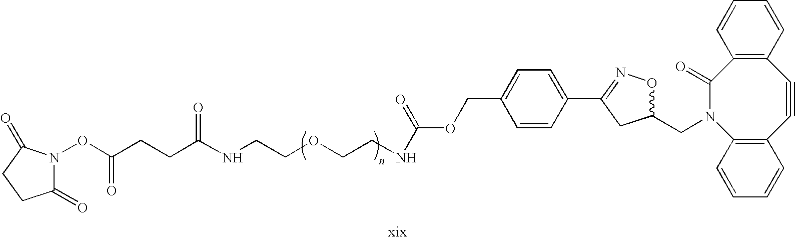

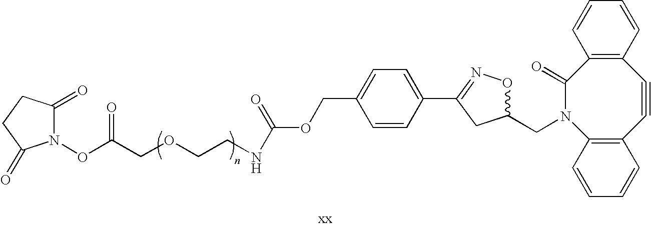

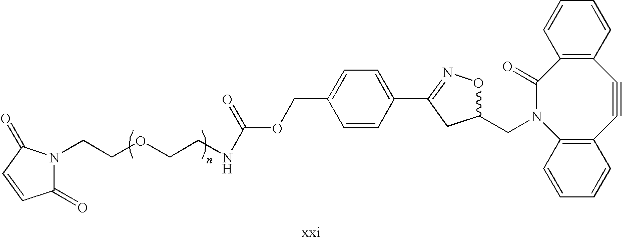

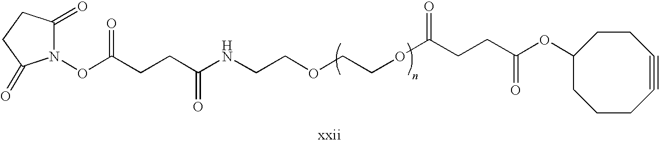

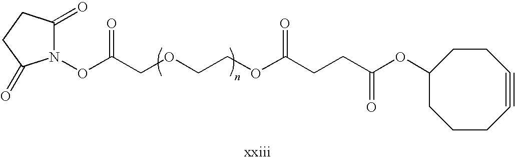

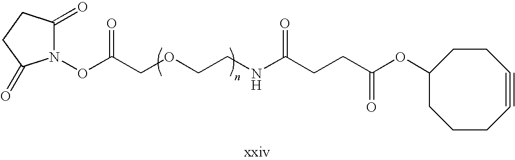

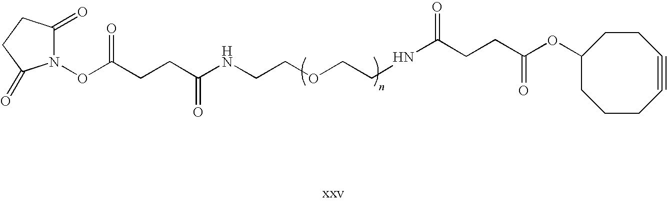

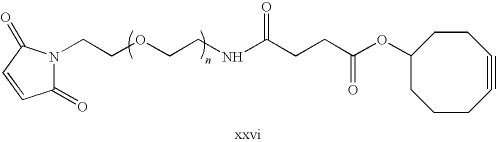

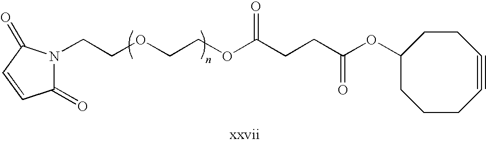

In certain embodiments, the present invention provides a polyplex comprising an

RNA, a polyanion, and a PEG-conjugated cationic polymer of formula III-a or a salt thereof:

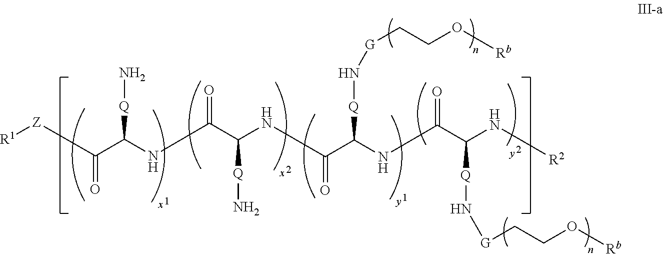

wherein each of R¹, Q, Z, G, x¹, x², n, R^{b} and R² is as defined above and as described in classes and subclasses herein, both singly and in combination;
   y¹ is 1-200;
   y² is 1-200.

In certain embodiments, y¹ is about 1 to about 200. In certain embodiments, y¹ is about 25. In other embodiments, y¹ is about 5. In certain embodiments, y¹ is about 10. In other embodiments, y¹ is about 15. In other embodiments , y¹ is about 20. In other embodiments y¹ is about 1 to about 25. In other embodiments, y¹ is about 50. According to yet another embodiment, y¹ is about 25-75. In other embodiments, y¹ is about 100. In other embodiments, y¹ is selected from 10±5, 15±5, 25±5, 50±5, 75±10, 100±10, or 125±10.

In certain embodiments , y² is about 1 to about 200. In certain embodiments, y² is about 25. In other embodiments, y² is about 5. In certain embodiments, y² is about 10. In other embodiments, y² is about 15. In other embodiments, y² is about 20. In other embodiments y² is about 1 to about 25. In other embodiments, y² is about 50. According to yet another embodiment, y² is about 25-75. In other embodiments, y² is about 100. In other embodiments, y² is selected from 10±5, 15±5, 25±5, 50±5, 75±10, 100±10, or 125±10.

In certain embodiments, the present invention provides a method for preparing for a PEG-conjugated polyplex comprising an RNA, a polyanion, and a cationic polymer of formula III-a or a salt thereof comprising the steps of:
(1) providing a polyplex comprising an RNA, a polyanion, and a cationic polymer of formula I-a;
(2) optionally adjusting the pH of the polyplex solution to pH 4.0-9.0
(3) conjugating a compound of formula IV to the polyplex by reaction of the electrophile of formula IV and at least one amine group of formula I-a.

D. Targeting Group Attachment

PEG-conjugated polyplexes comprising RNA, a polyanion, and a polycation, described herein can be modified to enable active cell-targeting to maximize the benefits of current and future therapeutic agents. Because these polyplexes typically possess diameters greater than 20 nm, they exhibit dramatically increased circulation time when compared to stand-alone drugs due to minimized renal clearance. This unique feature of nanovectors leads to selective accumulation in diseased tissue, especially cancerous tissue due to the enhanced permeation and retention effect (“EPR”). The EPR effect is a consequence of the disorganized nature of the tumor vasculature, which results in increased permeability of polymer therapeutics and drug retention at the tumor site. In addition to passive cell targeting by the EPR effect, these polyplexes are designed to actively target tumor cells through the chemical attachment of targeting groups to the polyplex periphery. The incorporation of such groups is most often accomplished through end-group functionalization of the PEG block using chemical conjugation techniques. Like viral particles, polyplexes functionalized with targeting groups utilize receptor-ligand interactions to control the spatial distribution of the polyplexses after administration, further enhancing cell-specific delivery of therapeutics. In cancer therapy, targeting groups are designed to interact with receptors that are over-expressed in cancerous tissue relative to normal tissue such as folic acid, oligopeptides, sugars, and monoclonal antibodies. See Pan, D.; Turner, J. L.; Wooley, K. L. Chem. Commun. 2003, 2400-2401; Gabizon, A.; Shmeeda, H.; Horowitz, A. T.; Zalipsky, S. Adv. Drug Deliv. Rev. 2004, 56, 1177-1202; Reynolds, P. N.; Dmitriev, I.; Curiel, D. T. Vector. Gene Ther. 1999, 6, 1336-1339; Derycke, A. S. L.; Kamuhabwa, A.; Gijsens, A.; Roskams, T.; De Vos, D.; Kasran, A.; Huwyler, J.; Missiaen, L.; de Witte, P. A. M. T J. Nat. Cancer Inst. 2004, 96, 1620-30; Nasongkla, N., Shuai, X., Ai, H.; Weinberg, B. D. P., J.; Boothman, D. A.; Gao, J. Angew. Chem. Int. Ed. 2004, 43, 6323-6327; Jule, E.; Nagasaki, Y.; Kataoka, K. Bioconj. Chem. 2003, 14, 177-186; Stubenrauch, K.; Gleiter, S.; Brinkmann, U.; Rudolph, R.; Lilie, H. Biochem. J. 2001, 356, 867-873; Kurschus, F. C.; Kleinschmidt, M.; Fellows, E.; Dornmair, K.; Rudolph, R.; Lilie, H.; Jenne, D. E. FEBS Lett. 2004, 562, 87-92; and Jones, S. D.; Marasco, W. A. Adv. Drug Del. Rev. 1998, 31, 153-170.

The R^{b} moiety can be used to attach targeting groups for cell specific delivery including, but not limited to, proteins, oliogopeptides, antibodies, monosaccarides, oligosaccharides, vitamins, or other small biomolecules. Such targeting groups include, but are not limited to monoclonal and polyclonal antibodies (e.g. IgG, IgA, IgM, IgD, IgE antibodies), sugars (e.g. mannose, mannose-6-phosphate, galactose), proteins (e.g. Transferrin), oligopeptides (e.g. cyclic and acylic RGD-containing oligopedptides), and vitamins (e.g. folate).

In other embodiments, the R^{b} moiety of any of Formulae III, III-a, III-b, or IV is conjugated to biomolecules which promote cell entry and/or endosomal escape. Such biomolecules include, but are not limited to, oligopeptides containing protein transduction domains such as the HIV Tat peptide sequence (GRKKRRQRRR) or oligoarginine (RRRRRRRRR). Oligopeptides which undergo conformational changes in varying pH environments such oligohistidine (HHHHH) also promote cell entry and endosomal escape.

Compounds having R^{b} moieties suitable for Click chemistry are useful for conjugating said compounds to biological systems or macromolecules such as proteins, viruses, and cells, to name but a few. The Click reaction is known to proceed quickly and selectively under physiological conditions. In contrast, most conjugation reactions are carried out using the primary amine functionality on proteins (e.g. lysine or protein end-group). Because most proteins contain a multitude of lysines and arginines, such conjugation occurs uncontrollably at multiple sites on the protein. This is particularly problematic when lysines or arginines are located around the active site of an enzyme or other biomolecule. Thus, in some embodiments the present invention provides a method of conjugating the R^{b} groups of a compound of Formulae III, III-a, III-b, or IV to a macromolecule via Click chemistry or metal free click chemistry.

According to one embodiment, the R^{b} moiety is an azide-containing group. According to another embodiment, the R^{b} moiety is an alkyne-containing group. In certain embodiments, the R^{b} moiety has a terminal alkyne moiety. In other embodiments, the R^{b} moiety is an alkyne moiety having an electron withdrawing group. Accordingly, in such embodiments, the R^{b} moiety is

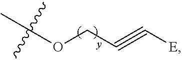

wherein E is an electron withdrawing group and y is 0-6. Such electron withdrawing groups are known to one of ordinary skill in the art. In certain embodiments, E is an ester. In other embodiments, the R^{b} moiety is

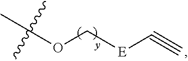

wherein E is an electron withdrawing group, such as a —C(O)O— group and y is 0-6.

In other embodiments, the R^{b} moiety is suitable for metal free click chemistry (also known as copper free click chemistry). Examples of such chemistries include cyclooctyne derivatives (Codelli, et. al. J. Am. Chem. Soc., 2008, 130, 11486-11493; Jewett, et. al. J. Am. Chem. Soc., 2010, 132, 3688-3690; Ning, et. al. Angew. Chem. Int. Ed., 2008, 47, 2253-2255), difluoro-oxanorbornene derivatives (van Berkel, et. al. Chem Bio Chem, 2007, 8, 1504-1508), or nitrile oxide derivatives (Lutz, et. al. Macromolecules, 2009, 42, 5411-5413). Without wishing to be bound by any particular theory, it is believed that the use of metal free click conditions offers certain advantages for the encapsulation of polynucleotides. Such functionalized PEG derivatives suitable for metal free click chemistry are described in detail in U.S. Ser. No. 13/045,996, filed Mar. 11, 2011, the entirety of which is hereby incorporated herein by reference.

Certain metal-free click moieties are known in the literature. Examples include 4-dibenzocyclooctynol (DIBO)

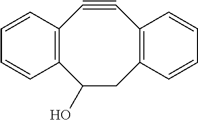

(from Ning et. al; Angew Chem Int Ed, 2008, 47, 2253); difluorinated cyclooctynes (DIFO or DFO)

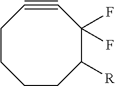

(from Codelli, et. al.; J. Am. Chem. Soc. 2008, 130, 11486-11493.); or biarylazacyclooctynone (BARAC)

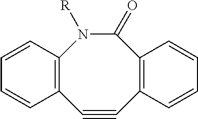

(from Jewett et. al.; J. Am. Chem. Soc. 2010, 132, 3688).

In certain embodiments, the present invention provides a targeted polyplex comprising an RNA, a polyanion, and a targeted PEG-conjugated cationic polymer of formula V or a salt thereof:

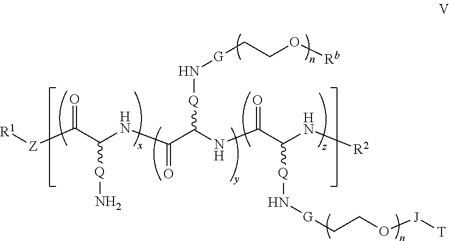

wherein each of R¹, Q, Z, G, x , y, n, R^{b} and R² is as defined above and as described in classes and subclasses herein, both singly and in combination;
   z is 1-200;
   each J is independently a valence bond or a bivalent, saturated or unsaturated, straight or branched C₁₋₁₂ hydrocarbon chain, wherein 0-6 methylene units of Q are independently replaced by -Cy-, —O—, —NH—, —S—, —OC(O)—, —C(O)O—, —C(O)—, —SO—, —SO₂—, —NHSO₂—, —SO₂NH—, —NHC(O)—, —C(O)NH—, —OC(O)NH—, or —NHC(O)O—, wherein:
      -Cy- is an optionally substituted 5-8 membered bivalent, saturated, partially unsaturated, or aryl ring having 0-4 heteroatoms independently selected from nitrogen, oxygen, or sulfur, or an optionally substituted 8-10 membered bivalent saturated, partially unsaturated, or aryl bicyclic ring having 0-5 heteroatoms independently selected from nitrogen, oxygen, or sulfur;
   each T is independently a targeting group.

It will be appreciated by one skilled in the art that the copolymer of formula V is a mixed, random copolymer comprised of side chain groups containing free amines or ammonium salts; conjugated PEG chains; and conjugated PEG chains with a terminal targeting group moiety. Furthermore, it is understood that x of formula V represents the number of free amines or ammonium salts; that y of formula V represents the number of repeats having pendant PEG chains; and that z of formula V represents the number of repeats that have a pendant PEG chain possessing a terminal targeting group.

In certain embodiments, z is about 1 to about 200. In certain embodiments, z is about 25. In certain embodiments, z is about 10. In certain embodiments, z is about 20. In certain embodiments, z is about 15. In other embodiments z is about 1 to about 25. In other embodiments, z is about 50. According to yet another embodiment, z is about 25-75. In other embodiments, z is about 100. In other embodiments, z is selected from 10±5, 15±5, 25±5, 50±5, 75±10, 100±10, or 125±10.

In certain embodiments, the J group is a valence bond as described above. In certain embodiments, the J group is a methylene group. In other embodiments, the J group is a carbonyl group. In certain embodiments, the J group of Formula V-a is a valence bond. In other embodiments, the J group is represented by a moiety in Table 4.

TABLE 4

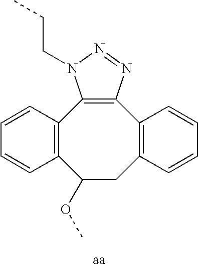

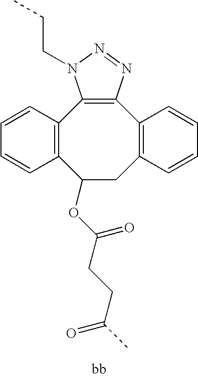

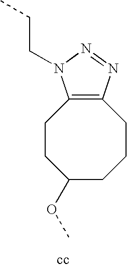

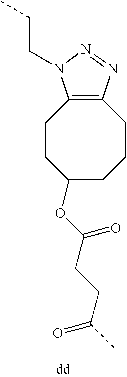

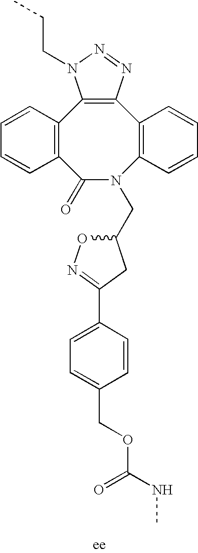

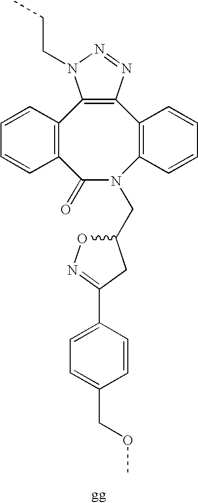

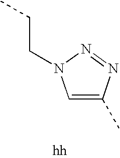

In certain embodiments, the present invention provides a method of preparation for a targeted PEG-conjugated polyplex comprising an RNA, a polyanion, and a cationic polymer of formula V or a salt thereof:

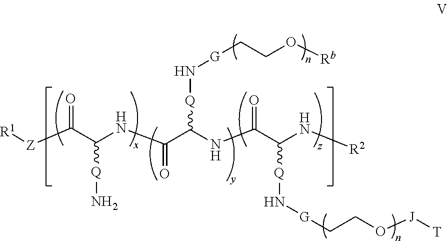

wherein each of R¹, Q, Z, G, x , y, z, n, J, T, R^{b} and R² is as defined above and as described in classes and subclasses herein, both singly and in combination;
   from a PEG-conjugated polyplex comprising an RNA, a polyanion, and a cationic polymer of formula III or a salt thereof:

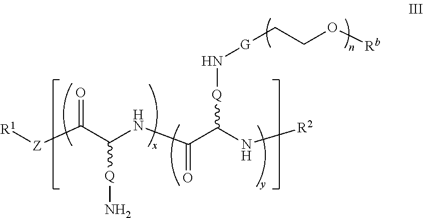

wherein each of R¹, Q, Z, G, x , y, n, R^{b} and R² is as defined above and as described in classes and subclasses herein, both singly and in combination;
   comprising the steps of:
(1) providing a PEG-conjugated polyplex comprising an RNA, a polyanion, and a cationic polymer of formula III;
(2) performing a Click reaction between the R^{b} group of formula III with a suitable click-ready targeting group to provide the targeted, PEG-conjugated polyplex of Formula V.

In certain embodiments, the present invention provides a targeted PEG-conjugated polyplex comprising an RNA, a polyanion, and a cationic polymer of formula V-a or a salt thereof:

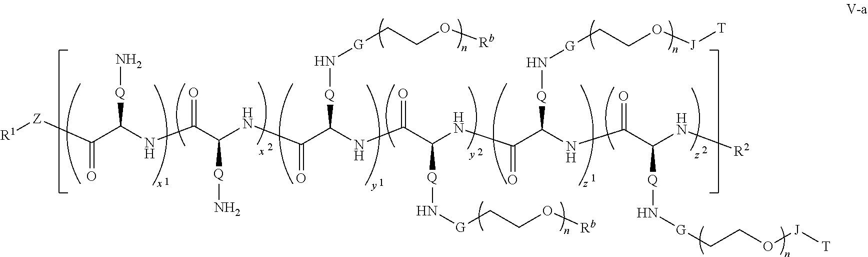

wherein each of R¹, Q, Z, G, x¹, x², y¹, y², z¹, z², n, J, T, R^{b} and R² is as defined above and as described in classes and subclasses herein, both singly and in combination.

In certain embodiments, the present invention provides a method of preparation for a targeted PEG-conjugated polyplex comprising an RNA, a polyanion, and a cationic polymer of formula V-a or a salt thereof:

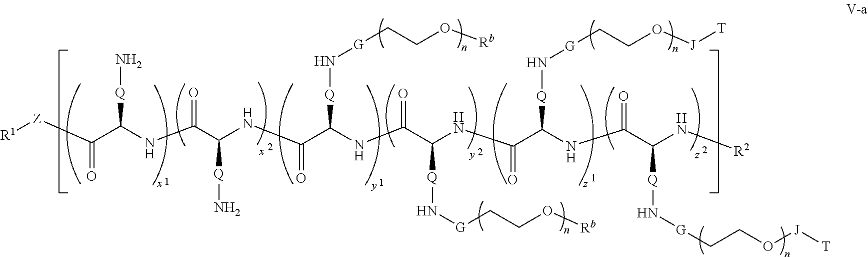

wherein each of R¹, Q, Z, G, x¹, x², y¹, y², z¹, z², n, J, T, R^{b} and R² is as defined above and as described in classes and subclasses herein, both singly and in combination;
   from a PEG-conjugated polyplex comprising an RNA, a polyanion, and a cationic polymer of formula III-a or a salt thereof:

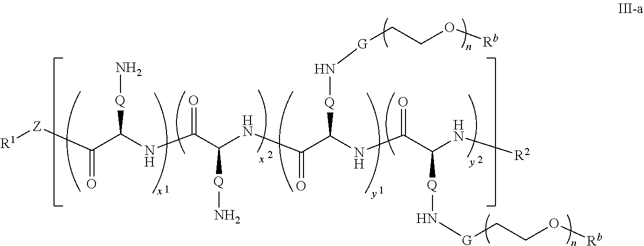

wherein each of R¹, Q, Z, G, x¹, x², y¹, y², n, R^{b} and R² is as defined above and as described in classes and subclasses herein, both singly and in combination;
   comprising the steps of:
(1) providing a PEG-conjugated polyplex comprising an RNA, a polyanion, and a cationic polymer of formula III-a; and
(2) performing a Click reaction between the R^{b} group of formula III-a with a suitable click-ready targeting group.

As generally described above, a suitable click-ready targeting group is comprised of a targeting group conjugated to a moiety capable of undergoing click chemistry. Such targeting groups are described in detail in United States patent application publication number 2009/0110662, published Apr. 30, 2009, the entirety of which is hereby incorporated by reference.

In certain embodiments, the present invention provides a targeted, PEG-conjugated polyplex comprising an RNA, a polyanion, and a cationic polymer of formula VI or a salt thereof:

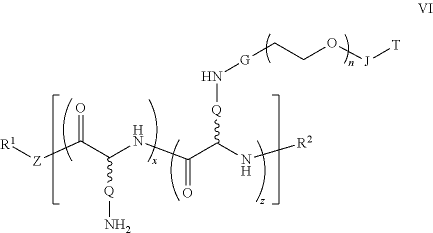

wherein each of R¹, Q, Z, G, x, z, n, J, T, and R² is as defined above and as described in classes and subclasses herein, both singly and in combination.

In certain embodiments, the present invention provides a method of preparing a PEG-conjugated polyplex comprising an RNA, a polyanion, and a cationic polymer of formula VI or a salt thereof:

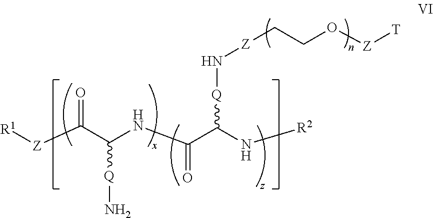

wherein each of R¹, Q, Z, G, x, z, n, J, T, and R² is as defined above and as described in classes and subclasses herein, both singly and in combination.
   from a compound of formula VII:

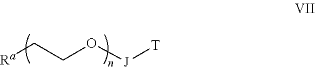

wherein each of R^{a}, J, n, and T is as defined above and as described in classes and subclasses herein, both singly and in combination;
   comprising the steps of:
(1) providing a polyplex comprising an RNA, a polyanion, and a cationic polymer of formula I;
(2) optionally adjusting the pH of the polyplex solution to pH 4.0-9.0; and
(3) conjugating the PEG to the polyplex by the reaction of the electrophile of formula VII and an amine group of formula I.

In certain embodiments, the present invention provides a targeted, PEG-conjugated polyplex comprising an RNA, a polyanion, and a cationic polymer of formula VI-a or a salt thereof:

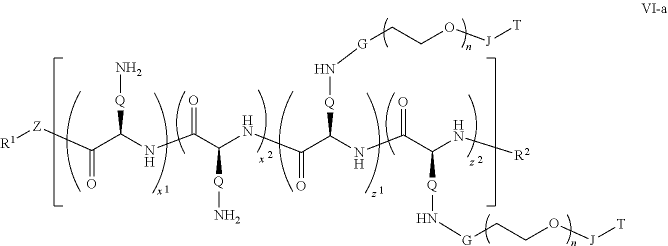

wherein each of R¹, Q, Z, G, x¹, x², z¹, z², n, J, T, and R² is as defined above and as described in classes and subclasses herein, both singly and in combination.

In certain embodiments, the present invention provides method of preparation for a PEG-conjugated polyplex comprising an RNA, a polyanion, and a cationic polymer of formula VI-a or a salt thereof:

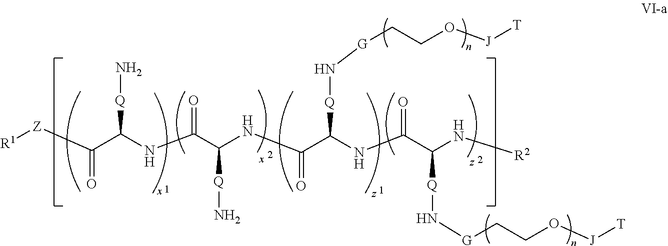

wherein each of R¹, Q, Z, G, x¹, x², z¹, z², n, J, T, and R² is as defined above and as described in classes and subclasses herein, both singly and in combination;
from a compound of formula VII:

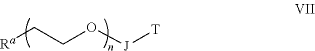

wherein each of R^{a}, J, n, and T is as defined above and as described in classes and subclasses herein, both singly and in combination
   comprising the steps of:
(1) providing a polyplex comprising an RNA, a polyanion, and a cationic polymer of formula I-a;
(2) optionally adjusting the pH of the polyplex solution to pH 4.0-9.0; and
(3) conjugating the PEG to the polyplex by the reaction of the electrophile of formula VII and an amine group of formula I-a.

### 4. Uses, Methods, and Compositions

As described herein, polyplexes of the present invention can encapsulate a wide variety of therpaeutic agents useful for treating a wide variety of diseases. In certain embodiments, the present invention provides a nucleotide-loaded polyplex, as described herein, wherein said polyplex is useful for treating the disorder for which the nucleotide is known to treat. According to one embodiment, the present invention provides a method for treating one or more disorders selected from pain, inflammation, arrhythmia, arthritis (rheumatoid or osteoarthritis), atherosclerosis, restenosis, bacterial infection, viral infection, depression, diabetes, epilepsy, fungal infection, gout, hypertension, malaria, migraine, cancer or other proliferative disorder, erectile dysfunction, a thyroid disorder, neurological disorders and hormone-related diseases, Parkinson's disease, Huntington's disease, Alzheimer's disease, a gastro-intestinal disorder, allergy, an autoimmune disorder, such as asthma or psoriasis, osteoporosis, obesity and comorbidities, a cognitive disorder, stroke, AIDS-associated dementia, amyotrophic lateral sclerosis (ALS, Lou Gehrig's disease), multiple sclerosis (MS), schizophrenia, anxiety, bipolar disorder, tauopothy, a spinal cord or peripheral nerve injury, myocardial infarction, cardiomyocyte hypertrophy, glaucoma, an attention deficit disorder (ADD or ADHD), a sleep disorder, reperfusion/ischemia, an angiogenic disorder, or urinary incontinence, comprising administering to a patient a PEG-conjugated polyplex, wherein said polyplex encapsulates a therapeutic agent suitable for treating said disorder.

In certain embodiments, the present invention provides a method for treating one or more disorders selected from autoimmune disease, an inflammatory disease, a metabolic disorder, a psychiatric disorder, diabetes, an angiogenic disorder, tauopathy, a neurological or neurodegenerative disorder, a spinal cord injury, glaucoma, baldness, or a cardiovascular disease, comprising administering to a patient an optionally targeted, PEG-covered polyplex wherein said polyplex encapsulates a therapeutic polynucleotide suitable for treating said disorder.

In certain embodiments, nucleotide-loaded polyplexes of the present invention are useful for treating cancer. Accordingly, another aspect of the present invention provides a method for treating cancer in a patient comprising administering to a patient an optionally targeted, PEG-covered polyplex wherein said polyplex encapsulates a therapeutic polynucleotide suitable for treating said cancer. In certain embodiments, the present invention relates to a method of treating a cancer selected from breast, ovary, cervix, prostate, testis, genitourinary tract, esophagus, larynx, glioblastoma, neuroblastoma, stomach, skin, keratoacanthoma, lung, epidermoid carcinoma, large cell carcinoma, small cell carcinoma, lung adenocarcinoma, bone, colon, adenoma, pancreas, adenocarcinoma, thyroid, follicular carcinoma, undifferentiated carcinoma, papillary carcinoma, seminoma, melanoma, sarcoma, bladder carcinoma, liver carcinoma and biliary passages, kidney carcinoma, myeloid disorders, lymphoid disorders, Hodgkin's, hairy cells, buccal cavity and pharynx (oral), lip, tongue, mouth, pharynx, small intestine, colon-rectum, large intestine, rectum, brain and central nervous system, and leukemia, comprising administering a polyplex in accordance with the present invention wherein said polyplex encapsulates a therapeutic polynucleotide suitable for treating said cancer.

Compositions

In certain embodiments, the invention provides a composition comprising a polyplex of this invention or a pharmaceutically acceptable derivative thereof and a pharmaceutically acceptable carrier, adjuvant, or vehicle. In certain embodiments, a composition of this invention is formulated for administration to a patient in need of such composition. In certain embodiments, a composition of this invention is formulated for oral administration to a patient.

The term “patient”, as used herein, means an animal, preferably a mammal, and most preferably a human.

The term “pharmaceutically acceptable carrier, adjuvant, or vehicle” refers to a non-toxic carrier, adjuvant, or vehicle that does not destroy the pharmacological activity of the polyplex with which it is formulated. Pharmaceutically acceptable carriers, adjuvants or vehicles that may be used in the compositions of this invention include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

Pharmaceutically acceptable salts of the polyplexes of this invention include those derived from pharmaceutically acceptable inorganic and organic acids and bases. Examples of suitable acid salts include acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, formate, fumarate, glucoheptanoate, glycerophosphate, glycolate, hemisulfate, heptanoate, hexanoate, hydrochloride, hydrobromide, hydroiodide, 2-hydroxyethanesulfonate, lactate, maleate, malonate, methanesulfonate, 2-naphthalenesulfonate, nicotinate, nitrate, oxalate, palmoate, pectinate, persulfate, 3-phenylpropionate, phosphate, picrate, pivalate, propionate, salicylate, succinate, sulfate, tartrate, thiocyanate, tosylate and undecanoate. Other acids, such as oxalic, while not in themselves pharmaceutically acceptable, may be employed in the preparation of salts useful as intermediates in obtaining the compounds of the invention and their pharmaceutically acceptable acid addition salts.

Salts derived from appropriate bases include alkali metal (e.g., sodium and potassium), alkaline earth metal (e.g., magnesium), ammonium and N+(C1-4 alkyl)4 salts. This invention also envisions the quaternization of any basic nitrogen-containing groups of the compounds disclosed herein. Water or oil-soluble or dispersible products may be obtained by such quaternization.

The compositions of the present invention may be administered orally, parenterally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. Preferably, the compositions are administered orally, intraperitoneally or intravenously. Sterile injectable forms of the compositions of this invention may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium.

For this purpose, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation.

In certain embodiments, the pharmaceutically acceptable compositions of this invention are formulated for oral administration. The pharmaceutically acceptable compositions of this invention may be orally administered in any orally acceptable dosage form including, but not limited to, capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavoring or coloring agents may also be added. In certain embodiments, pharmaceutically acceptable compositions of the present invention are enterically coated.

Alternatively, the pharmaceutically acceptable compositions of this invention may be administered in the form of suppositories for rectal administration. These can be prepared by mixing the agent with a suitable non-irritating excipient that is solid at room temperature but liquid at rectal temperature and therefore will melt in the rectum to release the drug. Such materials include cocoa butter, beeswax and polyethylene glycols.

The pharmaceutically acceptable compositions of this invention may also be administered topically, especially when the target of treatment includes areas or organs readily accessible by topical application, including diseases of the eye, the skin, or the lower intestinal tract. Suitable topical formulations are readily prepared for each of these areas or organs.

Topical application for the lower intestinal tract can be effected in a rectal suppository formulation (see above) or in a suitable enema formulation. Topically-transdermal patches may also be used.

For topical applications, the pharmaceutically acceptable compositions may be formulated in a suitable ointment containing the active component suspended or dissolved in one or more carriers. Carriers for topical administration of the polyplexes of this invention include, but are not limited to, mineral oil, liquid petrolatum, white petrolatum, propylene glycol, polyoxyethylene, polyoxypropylene compound, emulsifying wax and water. Alternatively, the pharmaceutically acceptable compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water.

For ophthalmic use, the pharmaceutically acceptable compositions may be formulated as micronized suspensions in isotonic, pH adjusted sterile saline, or, preferably, as solutions in isotonic, pH adjusted sterile saline, either with or without a preservative such as benzylalkonium chloride. Alternatively, for ophthalmic uses, the pharmaceutically acceptable compositions may be formulated in an ointment such as petrolatum.

The pharmaceutically acceptable compositions of this invention may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

The amount of the active ingredient and/or polyplex of the present invention that may be combined with the carrier materials to produce a composition in a single dosage form will vary depending upon the host treated, the particular mode of administration. Preferably, the compositions should be formulated so that a dosage of between 0.01-100 mg/kg body weight/day of the active ingredient and/or drug can be administered to a patient receiving these compositions.

It will be appreciated that dosages typically employed for the encapsulated drug are contemplated by the present invention. In certain embodiments, a patient is administered a drug-loaded polyplex of the present invention wherein the dosage of the drug is equivalent to what is typically administered for that drug. In other embodiments, a patient is administered a drug-loaded polyplex of the present invention wherein the dosage of the drug is lower than is typically administered for that drug.

It should also be understood that a specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease being treated. The amount of a compound of the present invention in the composition will also depend upon the particular compound in the composition.

In order that the invention described herein may be more fully understood, the following examples are set forth. It will be understood that these examples are for illustrative purposes only and are not to be construed as limiting this invention in any manner.

### EXAMPLE 1

### Synthesis of Asp(OBzl)NCA

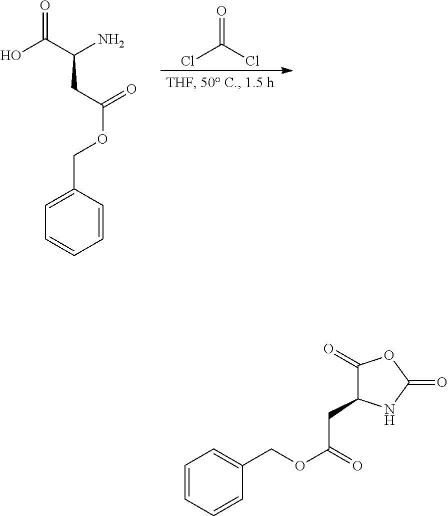

H-Asp(OBzl)-OH (14.0 g, 62.7 mmol) was suspended in 225 mL of anhydrous THF and heated to 50° C. Phosgene (20% in toluene) (40 mL, 80 mmol) was added to the amino acid suspension. The amino acid dissolved to give a clear solution over the course of approx. 15 min and was left reacting for another 25 min. The solution was concentrated on the rotovap, the white solid redissolved in a toluene/THF mixture (100 mL/50 mL) and the clear solution rotovaped to dryness. The white solid obtained was redissolved into 100 mL of THF, transferred to a beaker, and dry hexanes were added to precipitate the product. The white solid was isolated by filtration and rinsed twice with dry hexanes (2×200 mL) The NCA was isolated by filtration and dried in vacuo. 14.3 g (65% yield) of Asp(OBzl) NCA was isolated as a white solid. ¹H NMR (d₆-DMSO) δ 9.00 (1H), 7.48-7.25 (5H), 5.13 (2H), 4.69 (1H), 3.09 (1H), 3.09 (1H), 2.92 (1H) ppm

### EXAMPLE 2

### Synthesis of D-Asp(OBzl)NCA

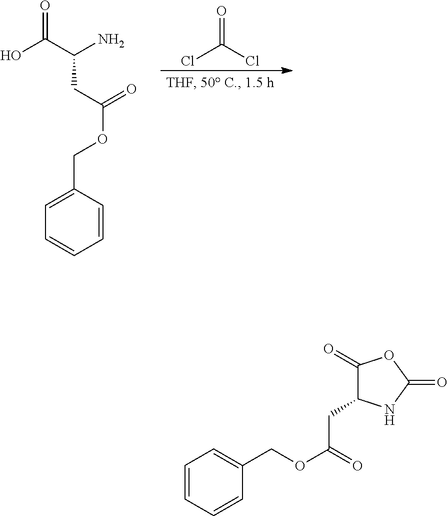

H-D-Asp(OBzl)-OH (30.0 g, 134 mmol) was suspended in 450 mL of anhydrous THF and heated to 50° C. Phosgene (20% in toluene) (100 mL, 100 mmol) was added the amino acid suspension. The amino acid dissolved over the course of approx. 50 min and was left reacting for another 30 min. The solution was concentrated on the rotovap, the white solid redissolved in a toluene/THF mixture (250 mL/50 mL) and the clear solution rotovaped to dryness. The white solid obtained was redissolved into 250 mL of THF, transferred to a beaker, and dry hexanes were added to precipitate the product. The white solid was isolated by filtration and rinsed twice with dry hexanes (2×400 mL) The NCA was isolated by filtration and dried in vacuo. 26.85 g (83.2% yield) of D-Asp(OBzl) NCA was isolated as a white solid. ¹H NMR (d₆-DMSO) δ 9.00 (1H), 7.48-7.25 (5H), 5.13 (2H), 4.69 (1H), 3.09 (1H), 2.92 (1H) ppm.

### EXAMPLE 3

### Preparation of Poly[DAsp(OBzl)-co-LAsp(OBzl)]-Ac

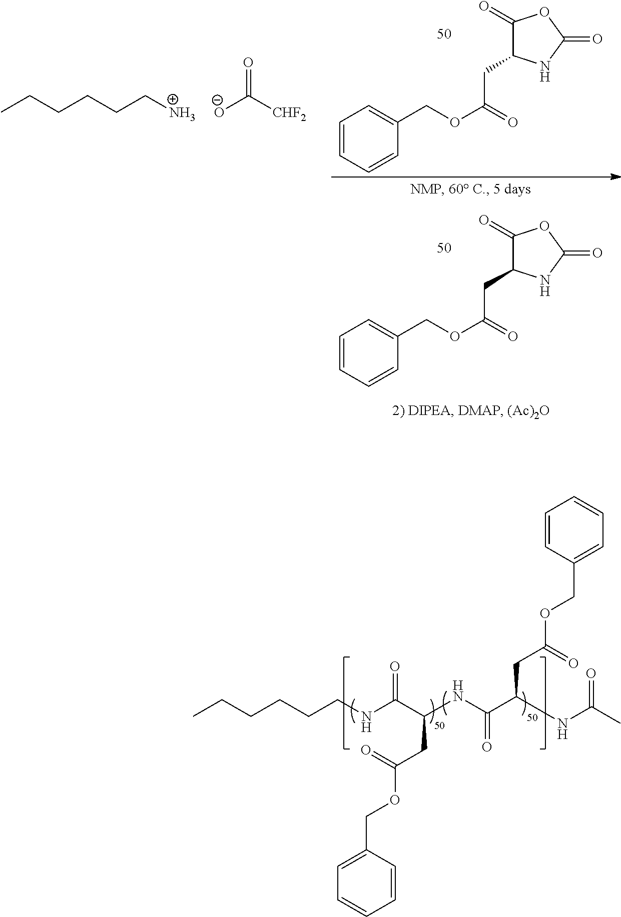

Poly(DLAsp(OBzl)) was synthesized as depicted in Scheme 2. A stock solution of hexylamine/DFA (0.5 M in NMP) was prepared. Asp(OBzl) NCA (9 g, 36.1 mmol), DAsp(OBzl) NCA (9 g, 36.1 mmol) were added to a 500 mL 2 neck flask, the flask was evacuated under reduced pressure, and subsequently backfilled with nitrogen gas (repeated twice). Dry N-methylpyrrolidone (NMP) (180 mL) was introduced by cannula, hexylamine/DFA (1.45 mL of stock solution) was syringed in and the solution was heated to 60° C. The reaction mixture was allowed to stir for 4 days at 60° C. under nitrogen gas until disappearance of the starting material by HPLC. The solution was cooled to room temperature and DIPEA (1.0 mL), DMAP (100 mg), and acetic anhydride (1.0 mL) were added. Stirring was continued for 1 hour at room temperature. The polymer was then placed in a 3500 g/mol molecular weight cut-off dialysis bag, dialyzed three times against methanol, three times against deionized water and freeze-dried. A white solid was obtained (7.2 g, 48.6% yield). ¹H NMR (d₆-DMSO) δ 8.61-7.95 (46H), 7.62-6.99 (263H), 5.25-4.79 (108H), 4.76-4.36 (50H), 3.02-2.71 (45H), 2.68-2.51 (39H), 1.86-1.72 (3H), 1.38-1.25 (2H), 1.25-1.08 (5H), 0.83-0.71 (3H) ppm. ¹³C NMR (d₆-DMSO) δ 170.11, 169.90, 135.80, 128.23, 127.83, 127.77, 65.68, 49.78, 35.82, 33.46, 33.07, 30.84, 28.72, 25.82, 24.52, 21.92 ppm, PDI=1.1 (DMF/THF GPC), Mn˜10,000 g/mol (MALDI-TOF MS).

### EXAMPLE 4

### Synthesis of Poly(DET)

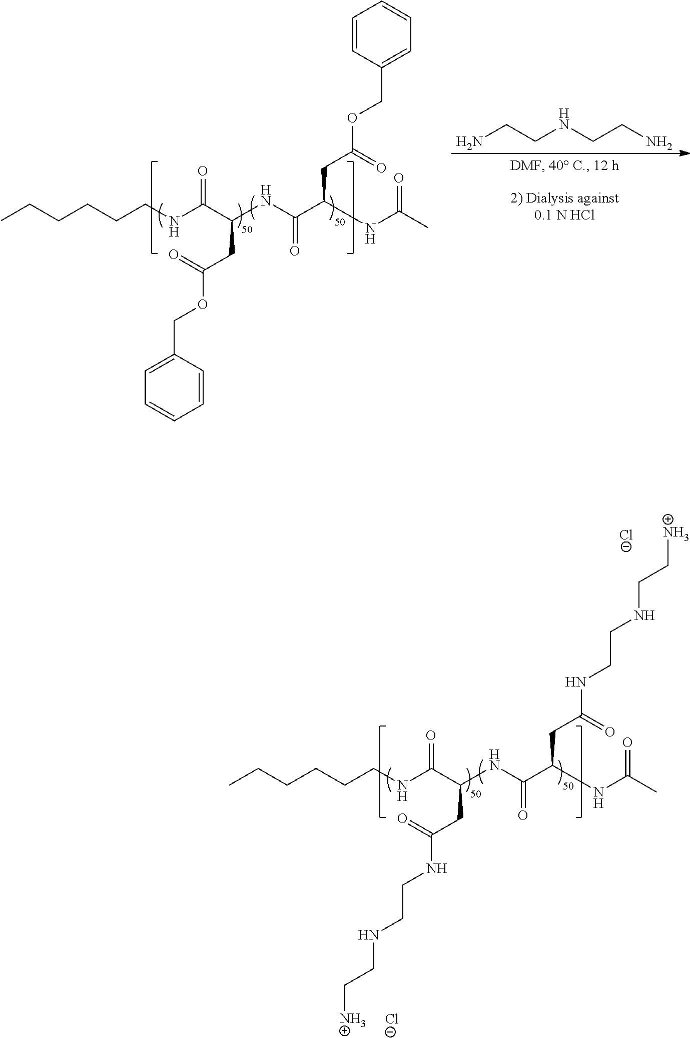

Poly(DLAsp(OBzl)) (2 g, 0.2 mmol) was introduced into an oven-dried two-neck flask and three vacuum/N₂ cycles were done. DET, (26 mL, 242 mmol) and dry DMF (26 mL) were syringed in the reaction flask. The reaction was stirred at 40° C. overnight under inert atmosphere. The reaction solution was then introduced into a 1,000 molecular weight cut-off dialysis bag and dialyzed three times against 0.1 M HCl and three times against deionized water. The solution was filtered through a 0.45 μm filter and the solution was freeze-dried. A highly hygroscopic white fluffy solid was recovered (0.4 g, 17% yield) ¹H NMR (D₂O) δ 4.28-3.97 (23H), 3.76-3.40 (447H), 3.38-3.14 (740H), 3.13-2.99 (254H), 2.99-2.61 (370H), 2.11-1.99 (4H), 1.92 (2H), 1.55-1.44 (2H), 1.35-1.22 (7H), 0.95-0.79 (3H) ppm.

### EXAMPLE 5

### Preparation of DNA Only Polyplexes

Poly(d/l Asp-DET)/DNA polyplexes were prepare by adding equal volumes of Poly(d/l Asp-DET) (From Example 4) solution (dissolved in dH₂O) and plasmid DNA solution (200 μg/mL in dH₂O) at N:P 10 ratio. Polymer was added to DNA solution, for a final volume of 200 μL, and incubated at room temperature for at least 30 minutes to allow polyplex formation. This solution was then ready for in vitro testing.

### EXAMPLE 6

### Preparation of siRNA Only Polyplexes

Poly(d/l Asp-DET)/siRNA polyplexes were prepare by adding equal volumes of Poly(d/l Asp-DET) (From Example 4) solution (dissolved in dH₂O) and siRNA solution (200 μg/mL in dH₂O) at N:P 10 ratio. Polymer was added to the siRNA solution, for a final volume of 200 μL, and incubated at room temperature for at least 30 minutes to allow polyplex formation. This solution was then ready for in vitro testing.

### EXAMPLE 7

### Preparation of Size-Controlled siRNA Containing Polyplexes

Poly(d/l Asp-DET)/DNA/siRNA polyplexes were prepare by combining equal volumes of plasmid DNA solution (200 μg/mL in dH2O), and siRNA solution (200 μg/mL in dH₂O), followed by the addition of poly(d/l Asp-DET) (From Example 4) solution (dissolved in dH₂O), at N:P 10 ratio. The resulting solution was incubated at room temperature for at least 30 minutes to allow polyplex formation. This solution was then ready for in vitro testing.

### EXAMPLE 8

### Gel Retardation Experiments

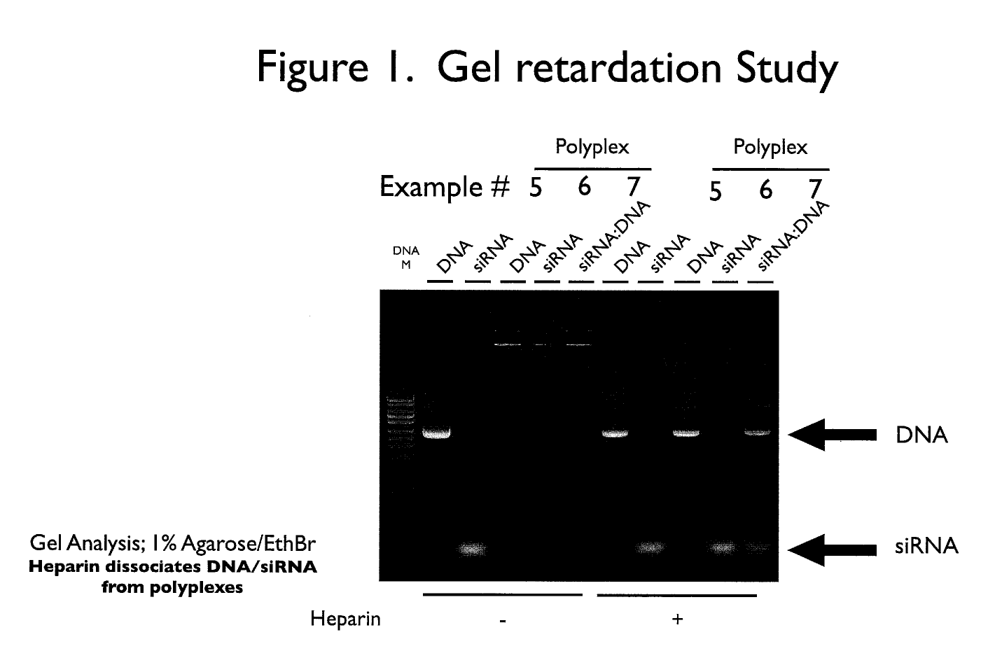

Gel loading dye was added to each polyplex from Example 5, Example 6, and Example 7 as well as control plasmid DNA and control siRNA. Samples (with and without the addition of heparin) were run on a 1% agarose/ethidium bromide gel in 1× TAE Buffer for 30 min at 200 V. FIG. 1 shows the agarose/ethidium bromide gel. This figure indicates that the polynucleotides from each of Example 5, Example 6, and Example 7 were effectively complexed and remain in the well. It can also be seen that no degradation occurred during the complexation step, because the samples run with heparin show the presence of intact, unchanged plasmid DNA and/or siRNA.

### EXAMPLE 9

### Polyplex Size Analysis by Dynamic Light Scattering

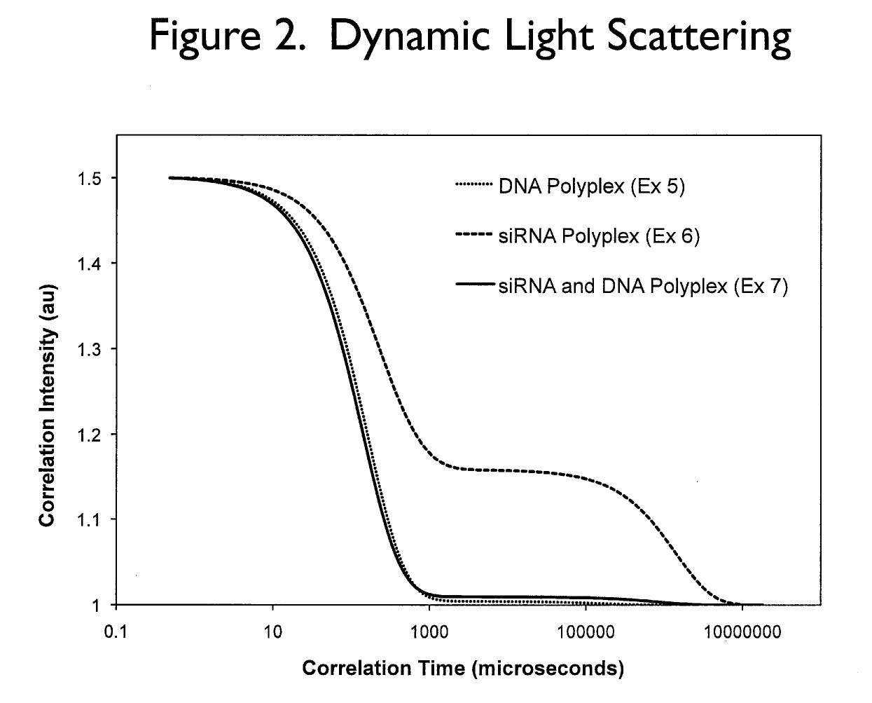

Dynamic Light Scattering analysis was performed using a DynaPro Dynamic Light Scattering Plate Reader (Wyatt Technology Corporation, Santa Barbara, Calif.). One hundred and twenty μL of each of Example 5, Example 6, and Example 7 was loaded into a 96 well plate and the correlation function recorded, shown in FIG. 2. The correlation function for Example 5 (DNA only) showed a uniform, singular decay representing a particle of ˜80 nm, while the correlation function for Example 6 (siRNA only) showed a multi-modal decay function representing a range of polyplex sizes, with the larger populations well over 1 micron in diameter. Surprisingly, the correlation function for Example 7 (siRNA with DNA) exhibits a uniform, singular decay function representing a particle of ˜80 nm. Thus, the size of the siRNA containing polyplex from Example 7 was effectively controlled through the use of a suitable polyanion.

### EXAMPLE 10

### Polyplex Size Analysis by Dynamic Light Scattering

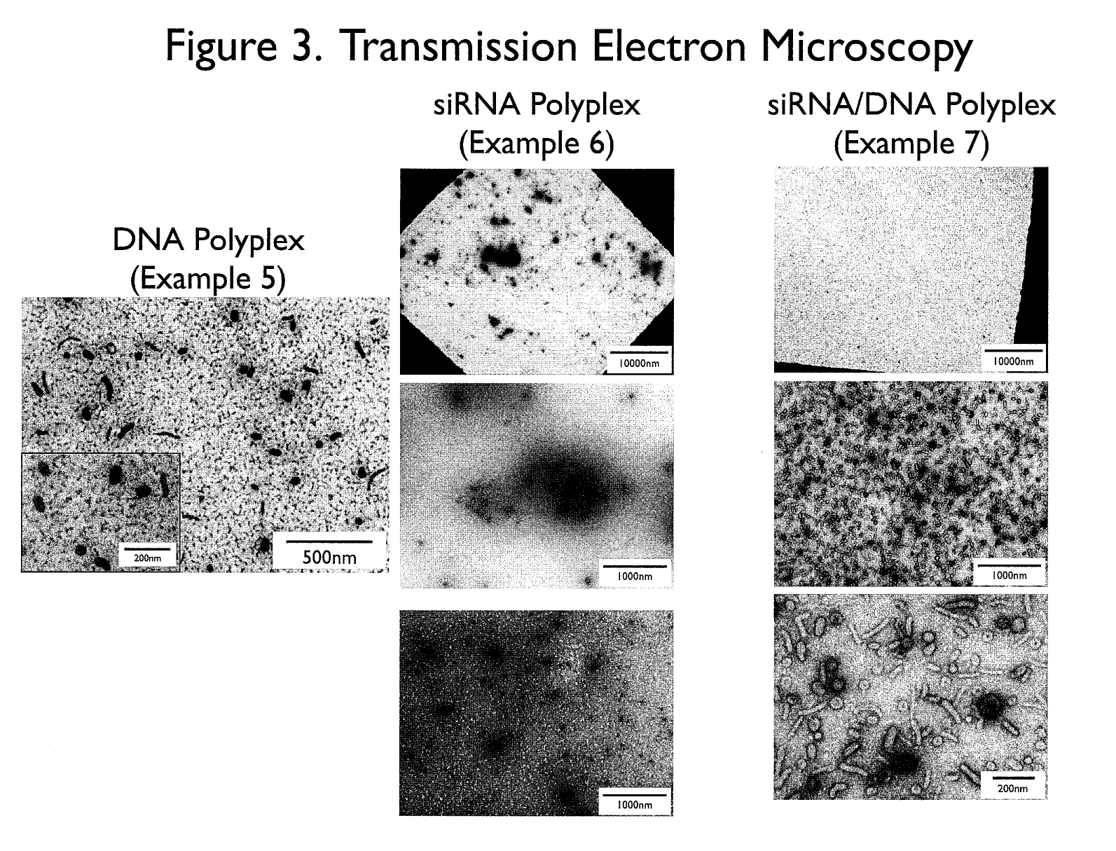

Five uL of each sample (Example 5, Example 6, and Example 7) was spotted onto formvar grids for 1-5 min, washed with H2O, incubated with 5% uranyl acetate for 1 min and washed again in H₂O. Images were taken using a Morgagni 268D electron microscope, FIG. 3. The images for Example 5 (DNA only) showed a uniform particle size of approximately 100 nm, while the images for Example 6 (siRNA only) showed a mixture of micron sized particles and particles on the ˜100 nm size scale. The micrographs for Example 7 (siRNA with DNA) also show particles with a uniform size of approximately 100 nm. These data confirm the dynamic light scattering results shown in Example 9.

### EXAMPLE 11

### Preparation of Bifunctional PEGs of the Present Invention

As described generally above, multiblock copolymers of the present invention are prepared using the heterobifunctional PEGs described herein and in U.S. Pat. No. 7,612,153, the entirety of which is hereby incorporated herein by reference. The preparation of multiblock polymers in accordance with the present invention is accomplished by methods known in the art, including those described in detail in U.S. Pat. No. 7,601,796, the entirety of which is hereby incorporated herein by reference.

While we have described a number of embodiments of this invention, it is apparent that our basic examples may be altered to provide other embodiments that utilize the polyplexes and methods of this invention. Therefore, it will be appreciated that the scope of this invention is to be defined by the appended claims rather than by the specific embodiments that have been represented by way of example.

1. A sub-micron particle comprising RNA, a suitable polyanion, and a suitable polycation.

2. The particle according to claim 1, wherein the polycation is a polyamine.

3. The particle according to claim 1, wherein the polycation is selected from poly(ketimines), poly(amino acids), poly(guanidinium), poly(alkylamines), poly(arylamines), poly(alkenylamines), and poly(alkynylamines), such as poly(imidazoles), poly(pyridines), poly(pyrimidines), poly(pyrazoles), poly(lysine), branched or linear poly(ethyleneimine), poly(histidine), poly(ornithine), poly(arginine), poly(asparginine), poly(glutamine), poly(tryptophan), poly(vinylpyridine), cationic guar gum, Oligofectamine® (from Invitrogen), polyfectamine® (from Qiagen), SuperFect® (from Qiagen), 293Fectin ((from Invitrogen), Cellfectin (from Invitrogen), DMRIE-C (from Invitrogen), FreeStyle (from Invitrogen), Lipofectamine 2000® (from Invitrogen), siPORT (from Invitrogen), Optifect (from Invitrogen), Neon (from Invitrogen), or salts and/or mixtures thereof.

4. The particle according to claim 3, wherein the polyanion is a polynucleotide.

5. The particle according to claim 3, wherein the polyanion is selected from polyelectrolytes, polyampholytes, poly(amino acids), poly(phosphonic acids), poly(phosphonates), poly(boronic acids), poly(boronates), polyphosphazines, double stranded DNA poly(styrene sulfonate), poly(acrylic acid), poly(acrylate), poly(aspartic acid), poly(glutamic acid), poly(aspartate), poly(glutamate), alginic acid, carboxymethylcellulose, alginates, poly(vinylbenzoate), poly(methacroic acid), polyphosphonates, poly(vinylphosphonic acid), or a salt and/or mixture thereof.

6. The particle according to claim 4, wherein the polyanion is DNA.

7. The particle according to claim 1, wherein the polycation is a compound of formula I, or a salt thereof: 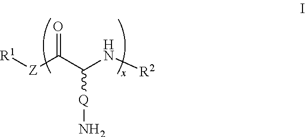
wherein:
x is 10-250;
each Q group is independently selected from a valence bond or a bivalent, saturated or unsaturated, straight or branched C₁₋₂₀ alkylene chain, wherein 0-9 methylene units of Q are independently replaced by -Cy-, —O—, —NH—, —S—, —OC(O)—, —C(O)O—, —C(O)—, —SO—, —SO₂—, —NHSO₂—, —SO₂NH—, —NHC(O)—, —C(O)NH—, —OC(O)NH—, or —NHC(O)O—, wherein:
-Cy- is an optionally substituted 5-8 membered bivalent, saturated, partially unsaturated, or aryl ring having 0-4 heteroatoms independently selected from nitrogen, oxygen, or sulfur, or an optionally substituted 8-10 membered bivalent saturated, partially unsaturated, or aryl bicyclic ring having 0-5 heteroatoms independently selected from nitrogen, oxygen, or sulfur;
Z is a valence bond or a bivalent, saturated or unsaturated, straight or branched C₁₋₁₂ hydrocarbon chain, wherein 0-6 methylene units of Q are independently replaced by -Cy-, —O—, —NH—, —S—, —OC(O)—, —C(O)O—, —C(O)—, —SO—, —SO₂—, —NHSO₂—, —SO₂NH—, —NHC(O)—, —C(O)NH—, —OC(O)NH—, or —NHC(O)O—, wherein:
-Cy- is an optionally substituted 5-8 membered bivalent, saturated, partially unsaturated, or aryl ring having 0-4 heteroatoms independently selected from nitrogen, oxygen, or sulfur, or an optionally substituted 8-10 membered bivalent saturated, partially unsaturated, or aryl bicyclic ring having 0-5 heteroatoms independently selected from nitrogen, oxygen, or sulfur;
R¹ is hydrogen, —N₃, —CN, a mono-protected amine, a di-protected amine, a protected aldehyde, a protected hydroxyl, a protected carboxylic acid, a protected thiol, a 9-30 membered crown ether, or an optionally substituted group selected from aliphatic, a 5-8 membered saturated, partially unsaturated, or aryl ring having 0-4 heteroatoms independently selected from nitrogen, oxygen, or sulfur, an 8-10 membered saturated, partially unsaturated, or aryl bicyclic ring having 0-5 heteroatoms independently selected from nitrogen, oxygen, or sulfur, or a detectable moiety or an oligopeptide targeting group;
R² is selected from hydrogen, an optionally substituted aliphatic group, an acyl group, a sulfonyl group, or a fusogenic peptide.

8. The particle according to claim 7, wherein the polyanion is DNA.

9. The particle according to claim 1, wherein the polycation is a compound of formula II or a salt thereof: 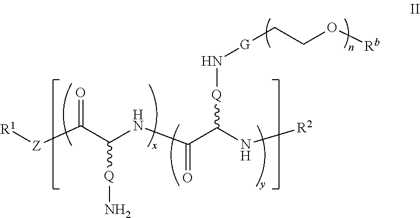
wherein:
x is 0-250;
y is 1-200;
each n is independently 10-1000;
Q is a valence bond or a bivalent, saturated or unsaturated, straight or branched C₁₋₁₈ alkylene chain, wherein 0-9 methylene units of Q are independently replaced by -Cy-, —O—, —NH—, —S—, —OC(O)—, —C(O)O—, —C(O)—, —SO—, —SO₂—, —NHSO₂—, —SO₂NH—, —NHC(O)—, —C(O)NH—, —OC(O)NH—, or —NHC(O)O—, wherein:
-Cy- is an optionally substituted 5-8 membered bivalent, saturated, partially unsaturated, or aryl ring having 0-4 heteroatoms independently selected from nitrogen, oxygen, or sulfur, or an optionally substituted 8-10 membered bivalent saturated, partially unsaturated, or aryl bicyclic ring having 0-5 heteroatoms independently selected from nitrogen, oxygen, or sulfur;
Z is a valence bond or a bivalent, saturated or unsaturated, straight or branched C₁₋₁₂ hydrocarbon chain, wherein 0-6 methylene units of Q are independently replaced by -Cy-, —O—, —NH—, —S—, —OC(O)—, —C(O)O—, —C(O)—, —SO—, —SO₂—, —NHSO₂—, —SO₂NH—, —NHC(O)—, —C(O)NH—, —OC(O)NH—, or —NHC(O)O—, wherein:
-Cy- is an optionally substituted 5-8 membered bivalent, saturated, partially unsaturated, or aryl ring having 0-4 heteroatoms independently selected from nitrogen, oxygen, or sulfur, or an optionally substituted 8-10 membered bivalent saturated, partially unsaturated, or aryl bicyclic ring having 0-5 heteroatoms independently selected from nitrogen, oxygen, or sulfur;
R¹ is hydrogen, —N₃, —CN, a mono-protected amine, a di-protected amine, a protected aldehyde, a protected hydroxyl, a protected carboxylic acid, a protected thiol, a 9-30 membered crown ether, or an optionally substituted group selected from aliphatic, a 5-8 membered saturated, partially unsaturated, or aryl ring having 0-4 heteroatoms independently selected from nitrogen, oxygen, or sulfur, an 8-10 membered saturated, partially unsaturated, or aryl bicyclic ring having 0-5 heteroatoms independently selected from nitrogen, oxygen, or sulfur, or a detectable moiety or an oligopeptide targeting group;
R² is selected from hydrogen, an optionally substituted aliphatic group, an acyl group, a sulfonyl group, or a fusogenic peptide;
R^{b} is —CH₃, a staturated or unsaturated alkyl moiety, an alkyne containing moiety, an azide containing moiety, a protected amine moiety, an aldehyde or protected aldehydes containing moiety, a thiol or protected thiol containing moiety, difluorocylcooctyne containing moiety, a nitrile oxide containing moiety, an oxanorbornadiene containing moiety, or an alcohol or protected alcohol containing moiety.

10. The particle according to claim 9, wherein the polyanion is DNA.

11. A targeted polyplex comprising an RNA, a polyanion, and a targeted PEG-conjugated cationic polymer of formula V or a salt thereof: 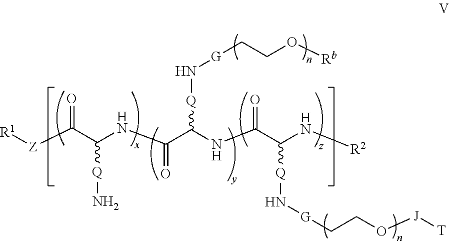
wherein:
x is 0-250;
y is 1-200;
z is 1-200;
each n is independently 10-1000;
each Q is a valence bond or a bivalent, saturated or unsaturated, straight or branched C₁₋₁₈ alkylene chain, wherein 0-9 methylene units of Q are independently replaced by -Cy-, —O—, —NH—, —S—, —OC(O)—, —C(O)O—, —C(O)—, —SO—, —SO₂—, —NHSO₂—, —SO₂—, —NHC(O)—, —C(O)NH—, —OC(O)NH—, or —NHC(O)O—, wherein:
-Cy- is an optionally substituted 5-8 membered bivalent, saturated, partially unsaturated, or aryl ring having 0-4 heteroatoms independently selected from nitrogen, oxygen, or sulfur, or an optionally substituted 8-10 membered bivalent saturated, partially unsaturated, or aryl bicyclic ring having 0-5 heteroatoms independently selected from nitrogen, oxygen, or sulfur;
each G is independently a valence bond or a bivalent, saturated or unsaturated, straight or branched C₁₋₁₂ hydrocarbon chain, wherein 0-6 methylene units of Q are independently replaced by -Cy-, —O—, —NH—, —S—, —OC(O)—, —C(O)O—, —C(O)—, —SO—, —SO₂—, —NHSO₂—, —SO₂NH—, —NHC(O)—, —C(O)NH—, —OC(O)NH—, or —NHC(O)O—, wherein:
-Cy- is an optionally substituted 5-8 membered bivalent, saturated, partially unsaturated, or aryl ring having 0-4 heteroatoms independently selected from nitrogen, oxygen, or sulfur, or an optionally substituted 8-10 membered bivalent saturated, partially unsaturated, or aryl bicyclic ring having 0-5 heteroatoms independently selected from nitrogen, oxygen, or sulfur;
each J is independently a valence bond or a bivalent, saturated or unsaturated, straight or branched C₁₋₁₂ hydrocarbon chain, wherein 0-6 methylene units of Q are independently replaced by -Cy-, —O—, —NH—, —S—, —OC(O)—, —C(O)O—, —C(O)—, —SO—, —SO₂—, —NHSO₂—, —SO₂NH—, —NHC(O)—, —C(O)NH—, —OC(O)NH—, or —NHC(O)O—, wherein:
-Cy- is an optionally substituted 5-8 membered bivalent, saturated, partially unsaturated, or aryl ring having 0-4 heteroatoms independently selected from nitrogen, oxygen, or sulfur, or an optionally substituted 8-10 membered bivalent saturated, partially unsaturated, or aryl bicyclic ring having 0-5 heteroatoms independently selected from nitrogen, oxygen, or sulfur;
each Z is a valence bond or a bivalent, saturated or unsaturated, straight or branched C₁₋₁₂ hydrocarbon chain, wherein 0-6 methylene units of Q are independently replaced by -Cy-, —O—, —NH—, —S—, —OC(O)—, —C(O)O—, —C(O)—, —SO—, —SO₂—, —NHSO₂—, —SO₂NH—, —NHC(O)—, —C(O)NH—, —OC(O)NH—, or —NHC(O)O—, wherein:
-Cy- is an optionally substituted 5-8 membered bivalent, saturated, partially unsaturated, or aryl ring having 0-4 heteroatoms independently selected from nitrogen, oxygen, or sulfur, or an optionally substituted 8-10 membered bivalent saturated, partially unsaturated, or aryl bicyclic ring having 0-5 heteroatoms independently selected from nitrogen, oxygen, or sulfur;
R¹ is hydrogen, —N₃, —CN, a mono-protected amine, a di-protected amine, a protected aldehyde, a protected hydroxyl, a protected carboxylic acid, a protected thiol, a 9-30 membered crown ether, or an optionally substituted group selected from aliphatic, a 5-8 membered saturated, partially unsaturated, or aryl ring having 0-4 heteroatoms independently selected from nitrogen, oxygen, or sulfur, an 8-10 membered saturated, partially unsaturated, or aryl bicyclic ring having 0-5 heteroatoms independently selected from nitrogen, oxygen, or sulfur, or a detectable moiety or an oligopeptide targeting group;
R² is selected from hydrogen, an optionally substituted aliphatic group, acyl group, sulfonyl group, or a fusogenic peptide;
R^{b} is —CH₃, a staturated or unsaturated alkyl moiety, an alkyne containing moiety, an azide containing moiety, a protected amine moiety, an aldehyde or protected aldehydes containing moiety, a thiol or protected thiol containing moiety, difluorocylcooctyne containing moiety, a nitrile oxide containing moiety, an oxanorbornadiene containing moiety, or an alcohol or protected alcohol containing moiety; and
T is a targeting group.

12. The polyplex according to claim 11, wherein the polyanion is DNA.

13. The particle according to claim 1, wherein the particle is substantially free of polypeptides, oligopeptides, and polynucleotides other than those encapsulated.

14. The particle according to claim 1, characterized in that the particle size ranges from 20-200 nm.

15. A pharmaceutically acceptable composition comprising a polyplex according to claim 1.
